(19) United States
(12) Patent Application Publication
(10) Pub. No.: US 2025/0101004 A1
(43) Pub. Date: Mar. 27, 2025

(54) NOVEL KALIRIN INHIBITORS FOR REGULATION OF SYNAPTIC PLASTICITY AND NEUROPATHIC PAIN TREATMENT

(71) Applicant: Northwestern University, Evanston, IL (US)

(72) Inventors: Euan Lachlan Parnell, Evanston, IL (US); Peter Penzes, Evanston, IL (US)

(21) Appl. No.: 18/726,646

(22) PCT Filed: Jan. 5, 2023
(86) PCT No.: PCT/US2023/060162

§ 371 Date: Jul. 3, 2024

Related U.S. Application Data
(60) Provisional application No. 63/266,459, filed on Jan. 5, 2022.

(51) Int. Cl. C07D 403/12; A61K 31/4045; A61K 31/416; A61K 31/4184; A61K 31/423; A61K 31/428; A61K 31/437; A61K 31/4439; A61K 31/454; C07D 209/18; C07D 209/42; C07D 401/14; C07D 413/12; C07D 417/12; C07D 471/04

(52) U.S. Cl. CPC C07D 403/12; A61K 31/4045; A61K 31/416; A61K 31/4184; A61K 31/423; A61K 31/428; A61K 31/437; A61K 31/4439; A61K 31/454; C07D 209/18; C07D 209/42; C07D 401/14; C07D 413/12; C07D 417/12; C07D 471/04

(57) ABSTRACT

Disclosed are substituted indole compounds and other substituted nitrogen-containing heteroaryl compounds. The disclosed compounds and compositions thereof may be utilized in methods for inhibiting kalirin, including methods for treating and/or preventing diseases or disorders associated with kalirin activity or expression such as neuropathic pain, chronic pain, and epilepsy.

### CROSS REFERENCE TO RELATED APPLICATIONS

This application claims the benefit of priority of U.S. Provisional Patent Application Ser. No. 63/266,459, filed Jan. 5, 2022, the contents of which is incorporated by reference in its entirety.

### STATEMENT REGARDING FEDERALLY SPONSORED RESEARCH OR DEVELOPMENT

This invention was made with government support under R01MH071316-02S1 awarded by the Department of Health and Human Services, National Institutes of Health, and National Institute of Mental Health. The government has certain rights in the invention.

### REFERENCE TO AN ELECTRONIC SEQUENCE LISTING

The contents of the electronic sequence listing (702581.02269.xml; Size: 9,719 bytes; and Date of Creation: Jan. 3, 2023) is herein incorporated by reference in its entirety.

### BACKGROUND

Neuropathic pain is caused by disorders of, or damage to, the nervous system. The affected nerves can induce the sensation of pain in the brain. Kalirin, a guanine exchange factor that activates the RAC-PAK pathway, has been found to be the most highly abundant in spines, and play key roles in spine plasticity. Kalirin has been directly implicated in CNS pathogenesis by genetic, postmortem neuropathological, and functional studies. Inhibiting kalirin using small-molecule compounds could block synaptic plasticity in the spinal dorsal horn, and may provide a valuable approach in regulating synaptic plasticity and treating neuropathic pain, chronic pain, and epilepsies. Kalirin could be an effective target because its expression is largely brain specific, its activity can be modulated by upstream pathways, and its inhibition reduces dendritic spine plasticity.

As such, there exists a need for compounds, compositions, and methods that inhibit kalirin. There also exists a need for compounds, compositions, and methods that inhibit the expression of kalirin and/or a biological activity of kalirin such as guanine-nucleotide exchange factor (GEF) activity.

### SUMMARY

Disclosed are compounds, compositions, and related methods of use for the inhibition of kalirin. The disclosed compounds, compositions, and methods can be utilized to treat diseases and disorders associated with kalirin activity such as neuropathic pain, chronic pain, and epilepsies.

The disclosed compounds may be described as substituted indole compounds and other substituted nitrogen-containing heteroaryl compounds. The disclosed compounds and compositions thereof may be utilized in methods for inhibiting kalirin and/or a biological activity of kalirin, including methods for treating and/or preventing diseases or disorders associated with kalirin activity or expression such as neuropathic pain, chronic pain, and epilepsies.

The disclosed compounds may be directed to a compound of the following formula or a salt or hydrate thereof:

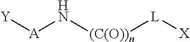

wherein:
Y is selected from hydrogen, alkyl, thiyl, haloalkyl, carboxamido, amido, amino, heteroaryl (e.g. pyridyl), alkylene-alkoxy, hydroxyalkyl, and heterocyclyl (e.g. piperidinyl);
A is an optionally substituted arylene (e.g. phenylene) or an optionally substituted heteroarylene selected from

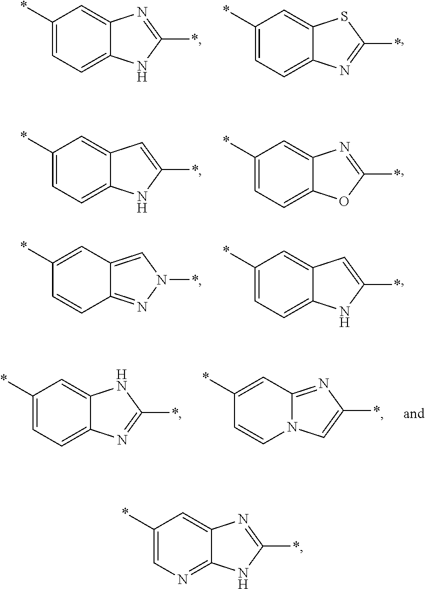

said substituent selected from hydroxyalkyl, halo, and alkyl;
n is 0 or 1;
L is a divalent linker selected from alkylene and halo substituted alkylene; and
X is an optionally substituted heteroaryl selected from

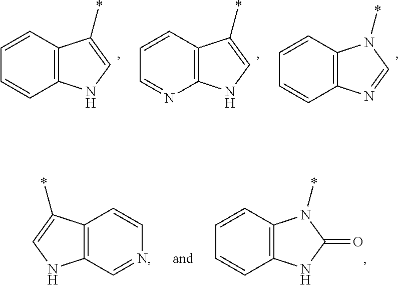

said substituent selected from halo and alkyl.

In some embodiments, the disclosed compounds may be directed to a compound as disclosed herein, wherein Y is sec-butyl or isopropyl, A is

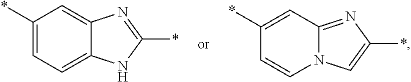

n is 1, L is methylene, and X is

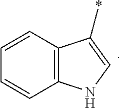 [SMILES: *c1c[nH]c2ccccc12]

The disclosed compounds and compositions may be utilized in methods for inhibition of kalirin or treatment and/or prevention of diseases and disorders associated with kalirin activity. Such methods can comprise administering to a subject in need thereof a pharmaceutical composition of a compound as disclosed herein.

In certain embodiments, the disclosed methods may be directed to treating and/or preventing neuropathic pain including, but not limited to, post herpetic neuralgia, reflex sympathetic dystrophy, cancer pain, phantom limb pain, entrapment neuropathy, peripheral neuropathy, and trigeminal neuralgia.

In certain embodiments, the disclosed methods may be directed to treating and/or preventing chronic pain including, but not limited to, headache, nerve damage pain, low back pain, arthritis pain, and fibromyalgia pain.

In certain embodiments, the disclosed methods may be directed to treating and/or preventing epilepsy.

### BRIEF DESCRIPTION OF THE FIGURES

FIG. 1 shows kalirin signaling within dendritic spines.

FIG. 2 shows GTPase activity schematic.

FIG. 3 shows overview of GEF/GTPase signaling pathways and selective effects of kalirin inhibition.

FIG. 4 shows that kalirin is sequestered to dendritic spines and governs their growth.

FIG. 5 shows expression of kalirin within spinal cord tissue.

FIG. 6 shows kalirin expression within the brain.

FIG. 7 demonstrates that alignment of closely related GEFs and GTPases reveals residues showing low homology (squares, homologous residues shown in shading). These residues map onto a distinct pocket outside of the catalytic site, and that form a deep drug binding pocket in the Kalirin/RAC1 interface composed of both RAC1 and Kalirin residues (crystal structure 5033 shown). DHPH alignments include kalirin (SEQ ID NO: 1), DBS (SEQ ID NO: 2), DBL (SEQ ID NO: 3) and Trio (SEQ ID NO: 4). GTPase alignments include CDC42 (SEQ ID NO: 5), RAC1 (SEQ ID NO: 6), RhoA (SEQ ID NO: 7) and Ras (SEQ ID NO: 8).

FIG. 8A-8C illustrate docked pose of compound isolated from in silico screening. FIG. 8A shows surface representation of kalirin-RAC1 interface, with docked pose of B04. FIG. 8B shows cartoon representation of A. FIG. 8C shows binding pose and interacting hydrogen bonded amino acids.

FIG. 9 demonstrates binding of compound to the catalytic domain of kalirin by a positive shift in thermodynamic properties as detected by fluorescence thermal shift.

FIG. 10 shows biochemical catalytic activity of kalirin is inhibited by compound but has no effect on the stability or intrinsic activity of RAC1 in the absence of kalirin's GEF domain (No GEF).

FIG. 11 illustrates that reaction rates from FIG. 10 were assessed and plotted against concentration.

FIG. 12 shows pharmokinetic profile of compound supports GI and BBB permeability desirable for in vivo application. Compound adheres to Lipinski rule of 5 and contains no pan assay interfering groups.

FIG. 13 shows cellular toxicity was measured using the cell-titre glow assay using dose response curves of compound alongside known RAC inhibitors and control toxic compound Patulin.

FIG. 14A-14D show cellular GEF assay reveals Cellular efficacy of B04 in line with biochemical data. Known compound, NSC23766 (FIGS. 14A and 14C), was run alongside B04 (FIGS. 14B and 14D) to confirm GEF inhibition (IC50 of NSC23766 matches published results).

FIG. 15A shows multi-electrode array analyses of the electrical activity of primary cultured neurons infected with synapsin-mcherry or synapsin-kalirin-mcherry.

FIG. 15B demonstrates that 24 wells of each infection was performed with equal titres of virus with 1 week of expression.

FIG. 15C shows that Weighted Mean Firing was assessed,

FIG. 15D shows that burst frequency was assessed.

FIG. 15E shows that network burst frequency was assessed.

FIG. 16A-16F show multi-electrode array analyses of the electrical activity of primary cultured neurons was assessed in the presence of compound B04, alongside the known RAC1 inhibitor NSC23776. Cells were treated for 4 hours with compound and firing rate (FIG. 16A) and burst frequency were found to be reduced (FIG. 16B). Dose response analysis confirmed activity of B04 (123 μM, FIG. 16D and FIG. +16F). However, NSC23766 (FIG. 16C and FIG. 16E) had 10× efficacy (7.4 μM) compared to previous results.

FIG. 17A demonstrates multi-electrode array analyses of the electrical activity of primary cultured neurons was assessed in the presence of compound B04, on neurons expressing mcherry or kalirin.

FIG. 17B shows that overexpression of kalirin conferred significant resistance to B04 treatment in terms of firing rate.

FIG. 17C shows that overexpression of kalirin conferred significant resistance to B04 treatment in terms of burst frequency.

FIG. 17D shows that overexpression of kalirin conferred significant resistance to B04 treatment in terms of network burst frequency.

FIG. 17E shows that overexpression of kalirin had no effect on network synchrony.

FIG. 18 shows summary of reference compounds and novel hit compounds.

FIG. 19A shows overlay of VAV2, pRex1 onto kalirin crystal structures suggest high structural homology within the catalytic site.

FIG. 19B demonstrates that the predicted binding site of B04 shows low sequence and structural homology, suggesting high potential for selective kalirin inhibition.

FIG. 20A shows modifiable moieties 1-4 of compound B04.

FIG. 20B shows the corresponding groups 1-4 obtained from modifying moieties 1-4 of compound B04.

FIG. 21 shows pilot crystallization screening yielded UV-positive crystals.

FIG. 22 shows a scheme illustrating (i) the role kalirin inhibitor plays in reducing acute and chronic pain and (ii) LTP drives spine density and neuronal activity via Kalirin/RAC activity.

FIG. 23 shows that no effect is observed on locomotion/anxiety at 60 mins (1-5 mg/kg). (*Center zone (28 cm×28 cm) activity is indirect parameter to analyze anxiety like agoraphobia.)

FIG. 24 illustrates a summary of analgesic efficacy in formalin pain model.

FIG. 25 shows a summary of analgesic efficacy in formalin pain model.

FIG. 26A shows alanine-scan through B04 binding site (e.g. D1343A, D1347A, S1360A).

FIG. 26B shows target selectivity and potential for selective GEF family inhibitor.

FIG. 27 shows CEREP44 mock data for various neuronal targets.

FIG. 28 shows the structure of compounds NW-01-0001 to NW-01-0020, including the structure of PP01B (i.e. compound NW-01-0012 or NW12; IUPAC name: 2-(1H-indol-3-yl)-N-[2-(propan-2-yl)-1H-imidazo[1,2-a]pyridine-5-yl]acetamide), which is a higher efficacy analog than PP01. Compound NW-01-0001 is also called NW01, PP01, or B04 throughout the specification.

FIGS. 29A and 29B demonstrate that PP01B (i.e. NW-01-0012 or NW12) shows suitability for in vitro cellular studies. FIG. 29A shows that HEK293T cells were treated with indicated compounds at varying doses (10-200 μM) for 24 hours. Cell viability was assessed by cell-titre glo assay. FIG. 29B shows selected compound LD50. Indicated values indicate the required concentration to reduce viability by 50%.

FIGS. 30A and 30B demonstrate efficacy of PP01 analogues at 4 and 24 hours, and recovery after 24 hour complete washout (washout). Indicated PP01 analogues were incubated with rat primary cultured cortical neurons at 50 μM to assess the ability to recapitulate PP01s ability to inhibit neuronal activity. Several compounds inhibit activity, but do not recover (e.g. NW006, FIG. 30A) suggesting toxic effects. However, NW12 (FIG. 30B) inhibited activity robustly, and recovered after drug washout, indicating a potential higher efficacy, reversible inhibitor of neuronal activity.

FIG. 31A, 31B show dose response curve of top PP01 analogues. FIG. 31A demonstrates that both NW07 and NW12 showed enhanced activity compared to PP01, with IC50s indicated. Both compounds showed reversible inhibition of neuronal activity (see FIGS. 30A and 30B). FIG. 31B shows structures of NW07 and NW12.

FIG. 31C demonstrates full dose response of top hit, NW12.

FIG. 32 shows the synthesis of compound NW-01-0001.

FIG. 33 demonstrates the synthesis of compound NW-01-0002.

FIG. 34 shows the synthesis of compound NW-01-0003.

FIG. 35 demonstrates the synthesis of compound NW-01-0004.

FIG. 36 illustrates the synthesis of compound NW-01-0005.

FIG. 37 shows the synthesis of compound NW-01-0006.

FIG. 38 illustrates the synthesis of compound NW-01-0007.

FIG. 39 demonstrates the synthesis of compound NW-01-0008.

FIG. 40 shows the synthesis of compound NW-01-0009.

FIG. 41 illustrates the synthesis of compound NW-01-0010.

FIG. 42 shows the synthesis of compound NW-01-0011.

FIG. 43 demonstrates the synthesis of compound NW-01-0012.

FIG. 44 shows the synthesis of compound NW-01-0013.

FIG. 45 illustrates the synthesis of compound NW-01-0014.

FIG. 46 demonstrates the synthesis of compound NW-01-0015.

FIG. 47 shows the synthesis of compound NW-01-0016.

FIG. 48 demonstrates the synthesis of compound NW-01-0017.

FIG. 49 illustrates the synthesis of compound NW-01-0018.

FIG. 50 shows the synthesis of compound NW-01-0019.

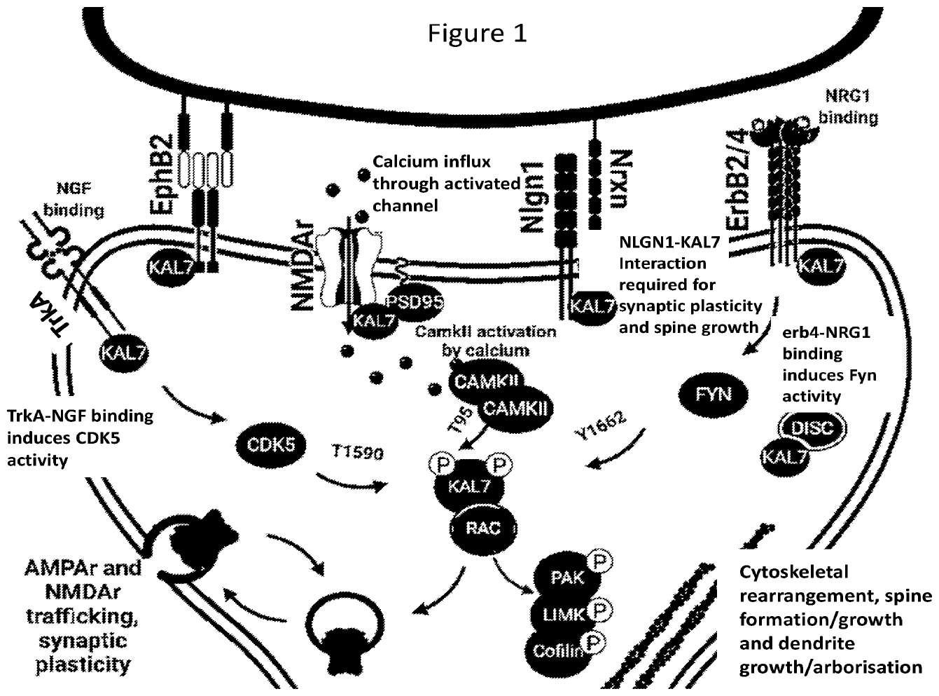

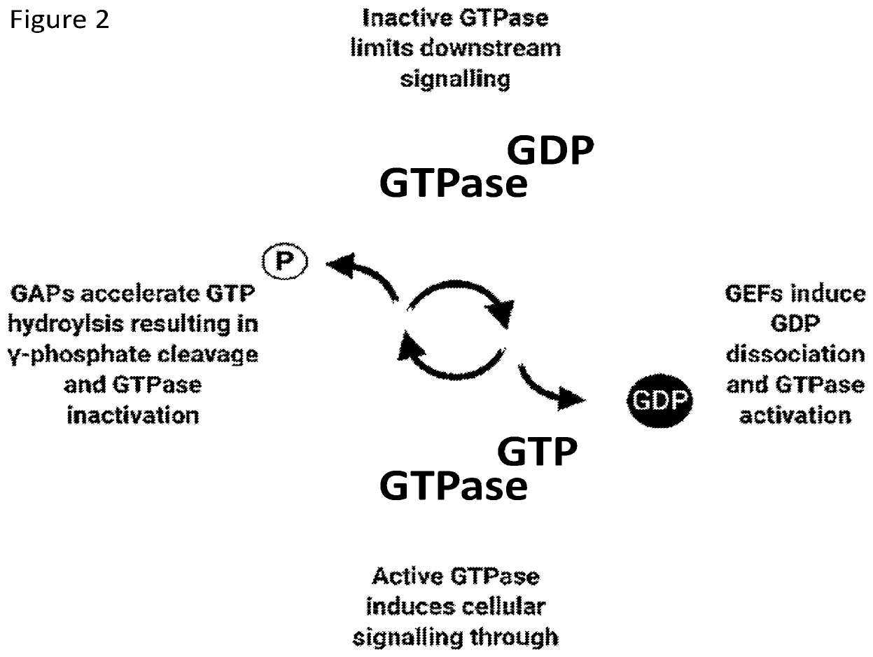

### DETAILED DESCRIPTION

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the claimed subject matter and does not pose a limitation on the scope of the claimed subject matter.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, a “subject in need thereof” may include a human and/or non-human animal. A “subject in need thereof” may include a subject having a disease, disorder, or condition associated with kalirin activity. A “subject in need thereof” may include a subject having neuropathic pain, including but not limited to, post herpetic neuralgia, reflex sympathetic dystrophy, cancer pain, phantom limb pain, entrapment neuropathy, peripheral neuropathy, trigeminal neuralgia, and any combinations thereof. A “subject in need thereof” may also include a subject having chronic pain, including but not limited to, headache, nerve damage pain, low back pain, arthritis pain, fibromyalgia pain, and any combinations thereof. A “subject in need thereof” may also include a subject having epilepsy.

The pharmaceutical compositions for use according to the methods as disclosed herein may include a combination of compounds as active ingredients. For example, the methods disclosed herein may be practiced using a composition containing one or more compounds that are kalirin inhibitors.

In some embodiments, the disclosed methods may be practiced by administering a first pharmaceutical composition (e.g., a pharmaceutical composition comprising a kalirin inhibitor) and administering a second pharmaceutical composition (e.g., a pharmaceutical composition comprising a kalirin inhibitor), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The compound as disclosed herein that is utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the compounds utilized in the methods disclosed herein may be formulated as a pharmaceutical composition in liquid form (e.g., an injectable liquid or gel).

The compound as disclosed herein that is utilized in the methods disclosed herein may be formulated as a pharmaceutical composition that includes an excipient, carrier, or diluent. For example, the excipient, carrier, or diluent may be selected from the group consisting of proteins, carbohydrates, sugar, talc, magnesium stearate, cellulose, calcium carbonate, and starch-gelatin paste.

The compound as disclosed herein that is utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The disclosed pharmaceutical compositions also may include disintegrants. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The disclosed pharmaceutical compositions also may include effervescent agents. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compound as disclosed herein may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### Chemical Entities

New chemical entities and uses for chemical entities are disclosed herein. The chemical entities may be described using terminology known in the art and further discussed below.

As used herein, a dash “−” an asterisk “*” or a plus sign “+” may be used to designate the point of attachment for any radical group or substituent group.

As used herein, the term “alkyl” refers to a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

As used herein, the term “alkylene” refers to a diradical of straight-chain or branched alkyl group (i.e., a diradical of straight-chain or branched C₁-C6 alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

As used herein, the term “halo” refers to a halogen substitution (e.g., —F, —Cl, —Br, or —I). The term “haloalkyl” refers to an alkyl group that is substituted with at least one halogen. For example, —CH₂F, —CHF₂, —CF₃, —CH₂CF₃, —CF₂CF₃, and the like.

As used herein, the term “arylene” refers to a diradical of an aryl group. The term “aryl” is art-recognized and refers to a carbocyclic aromatic group. Representative aryl groups include phenyl, naphthyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls.

As used herein, the term “heteroarylene” refers to a diradical of a heteroaryl group. The term “heteroaryl” is art-recognized and refers to a heterocyclic aromatic group. Representative heteroaryl groups include pyridinyl, quinolinyl, furanyl, thionyl, and the like. The term “heteroaryl” includes polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. In certain embodiments, the heteroaryl group is a 6-10 membered ring structure.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position. In one embodiment, the heterocyclyl is piperidinyl.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively

The term “thiyl” is art-recognized and refers to an alkyl group, as defined above, having a sulfur radical attached thereto. Representative thiyl groups include

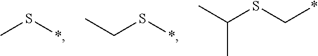

and the like.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

The term “carboxamido” as used herein refers to the radical —C(O)NRR′, where R and R′ may be the same or different. R and R′, for example, may be independently hydrogen, alkyl, aryl, arylalkyl, cycloalkyl, formyl, haloalkyl, heteroaryl, or heterocyclyl.

The term “amide” or “amido” or “amidyl” as used herein refers to a radical of the form —N(R¹)C(O)R²—, wherein R¹ and R² are each independently hydrogen, alkyl, alkoxy, amide, amino, aryl, arylalkyl, cycloalkyl, halogen, haloalkyl, heteroaryl, heterocyclyl, or hydroxyl.

The term “pyridyl” refers to a radical derived from pyridine, wherein the point of attachment to the pyridyl group could be any carbon on the pyridine ring.

The term “hydroxyalkyl” refers to a radical of the form —ROH, wherein R is an alkylene.

The term “piperidinyl” refers to a radical derived from piperidine, wherein the point of attachment to the piperidinyl group could be any atom (carbon or nitrogen atom) on the piperidine ring.

As used herein, “salt” refers to acid addition salts and basic addition salts. It may also refer to those salts that may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The compounds of the disclosure may be isomeric. In some embodiments, the disclosed compounds may be isomerically pure, wherein the compounds represent greater than about 99% of all compounds within an isomeric mixture of compounds. Also contemplated herein are compositions comprising, consisting essentially of, or consisting of an isomerically pure compound and/or compositions that are isomerically enriched, which compositions may comprise, consist essential of, or consist of at least about 50%, 60%, 70%, 80%, 90%, 95%, 96%, 97%, 98%, 99%, or 100% of a single isomer of a given compound.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the chiral or stereogenic carbon atom and or the optical rotation observed. The disclosed compounds encompass various stereo isomers and mixtures thereof. Stereoisomers include enantiomers and diastereomers. Mixtures of enantiomers or diastereomers may be designated (±)″ in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound and/or compositions that are enantiomer enriched, which compositions may comprise, consist essential of, or consist of at least about 50%, 60%, 70%, 80%, 90%, 95%, 96%, 97%, 98%, 99%, or 100% of a single enantiomer of a given compound (e.g., at least about 95% of an R enantiomer of a given compound).

In some embodiments, the disclosed subject matter relates to the compound as disclosed herein, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable aqueous or nonaqueous solutions, dispersions, suspensions or emulsions, whether or not sterile. Amounts of a compound effective inhibit kalirin may be determined empirically, and making such determinations is within the skill in the art.

It is understood by those skilled in the art that dosage amount will vary with the activity of a particular inhibitor or inactivator compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

Methods of preparing pharmaceutical formulations or compositions include the step of bringing an inhibitor or inactivator compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the disclosed compounds may be utilized as inhibitor or inactivator compounds for the manufacture of a medicament for therapeutic use in the treatment or inhibition of a disease or disorder associated with kalirin activity. Suitable diseases, disorders, or conditions may include (i) neuropathic pain, which may include, but is not limited to post herpetic neuralgia, reflex sympathetic dystrophy, cancer pain, phantom limb pain, entrapment neuropathy, peripheral neuropathy, trigeminal neuralgia, and any combinations thereof, (ii) chronic pain, which may include, but is not limited to headache, nerve damage pain, low back pain, arthritis pain, fibromyalgia pain, and any combinations thereof, and (iii) epilepsy.

Generally, with respect to various embodiments, the disclosed subject matter can be directed to method(s) for the treatment of neuropathic pain, chronic pain, and epilepsy. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

According to some embodiments, the treated subject may be a mammalian subject. Although the methods disclosed herein are particularly intended for the treatment of neuropathic pain, chronic pain, and epilepsy in humans, other mammals are included. By way of non-limiting examples, mammalian subjects include monkeys, equines, cattle, canines, felines, mice, rats and pigs.

The terms “treat, treating, treatment” as used herein and in the claims mean ameliorating one or more clinical indicia of disease activity in a subject having a pathologic disorder. “Treatment” refers to therapeutic treatment. Those in need of treatment are mammalian subjects suffering from any pathologic disorder. By “patient” or “subject in need” is meant any mammal for which administration of a compound or any pharmaceutical composition of the sort described herein is desired, in order to prevent, overcome, modulate or slow down such infliction. To provide a “preventive treatment” or “prophylactic treatment” is acting in a protective manner, to defend against or prevent something, especially a condition or disease.

More generally, the disclosed methods may be directed to affecting, modulate, reducing, inhibiting and/or preventing the initiation, and/or progression of neuropathic pain, chronic pain, and epilepsy associated with accumulation of kalirin.

### ILLUSTRATIVE EMBODIMENTS

The following Embodiments are illustrative and should not be interpreted to limit the scope of the claimed subject matter.

Embodiment 1. A compound of the following formula or a salt or hydrate thereof:

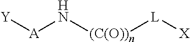

wherein:
Y is selected from hydrogen, alkyl, thiyl, haloalkyl, carboxamido, amido, amino, heteroaryl (e.g. pyridyl), alkylene-alkoxy, hydroxyalkyl, and heterocyclyl (e.g. piperidinyl);
A is an optionally substituted arylene (e.g. phenylene) or an optionally substituted heteroarylene selected from

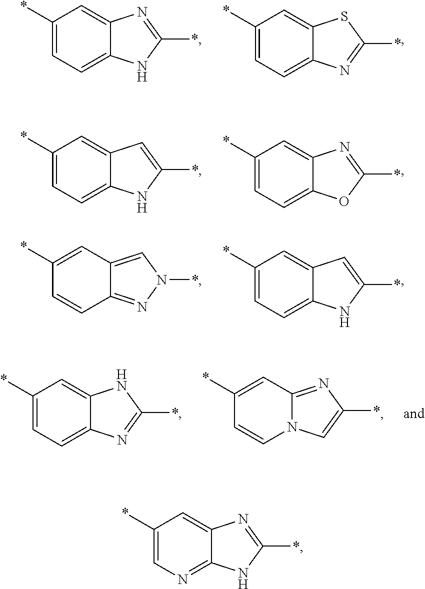

said substituent selected from hydroxyalkyl, halo, and alkyl;
n is 0 or 1;
L is a divalent linker selected from alkylene and halo substituted alkylene;
X is an optionally substituted heteroaryl selected from

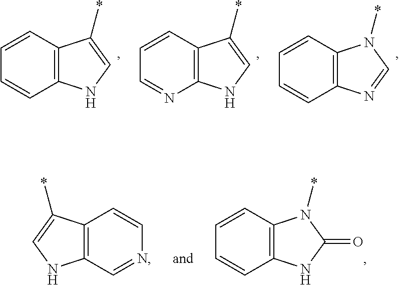

said substituent selected from halo and alkyl.

Embodiment 2. The compound of embodiment 1, wherein Y is sec-butyl or isopropyl, A is

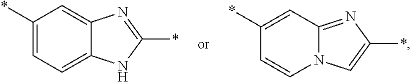

n is 1, L is methylene, and X is

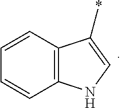 [SMILES: *c1c[nH]c2ccccc12]

Embodiment 3. The compound of embodiment 1 or 2, wherein L is selected from methylene (—CH₂—), ethylene (—CH₂—CH₂—),

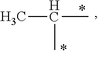

### and —CF₂—.

Embodiment 4. The compound of embodiment 1 of a formula selected from

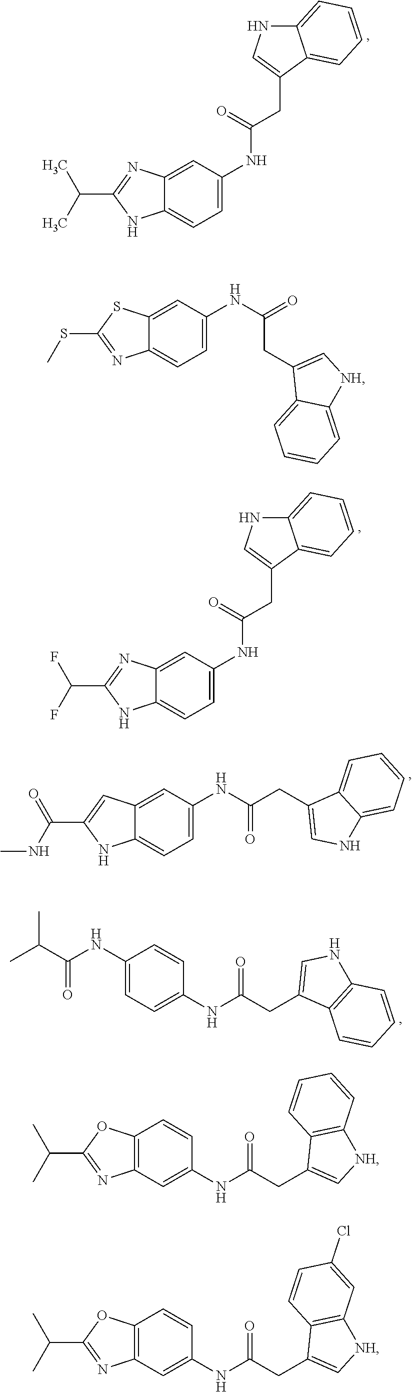

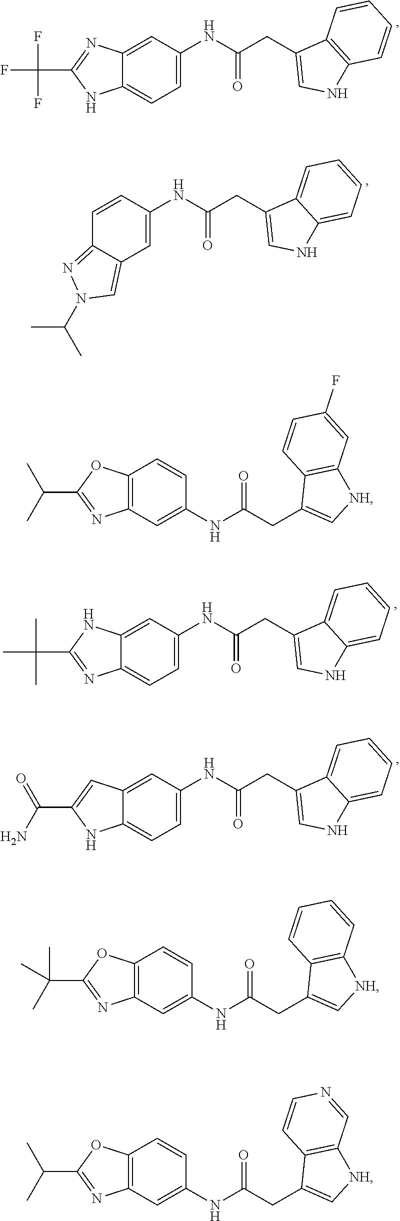

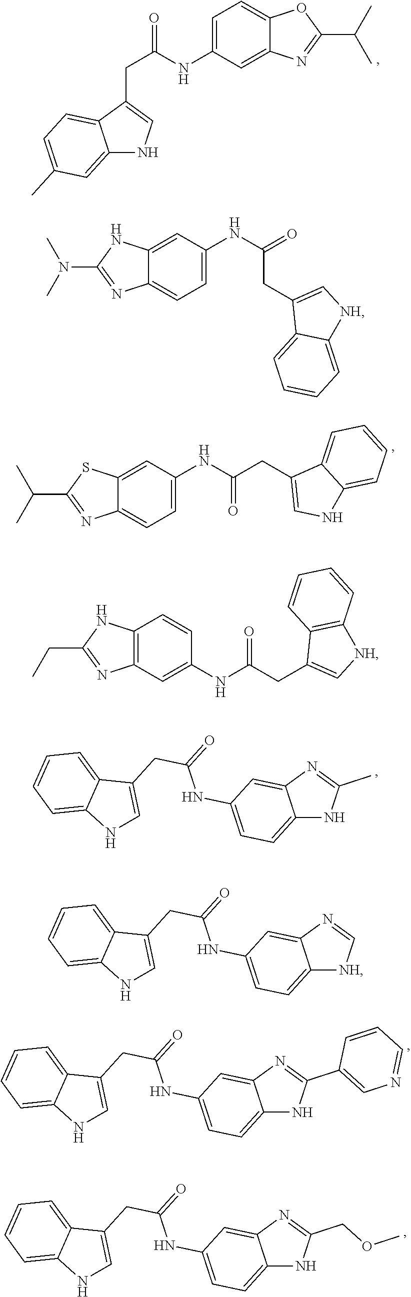

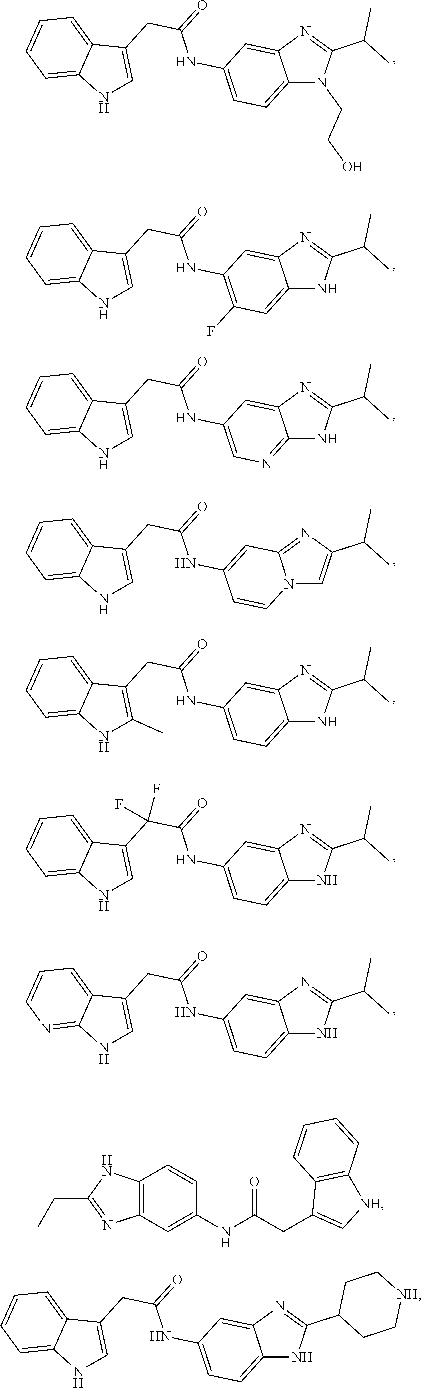

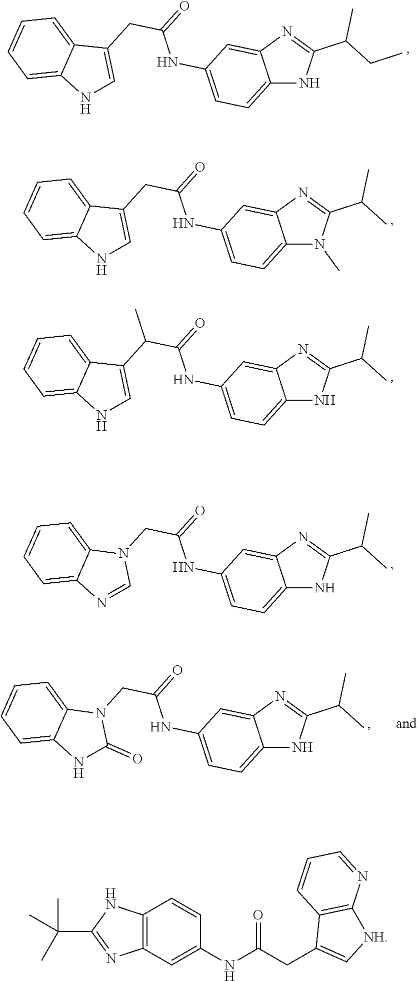

Embodiment 5. The compound of embodiment 1 of a formula selected from

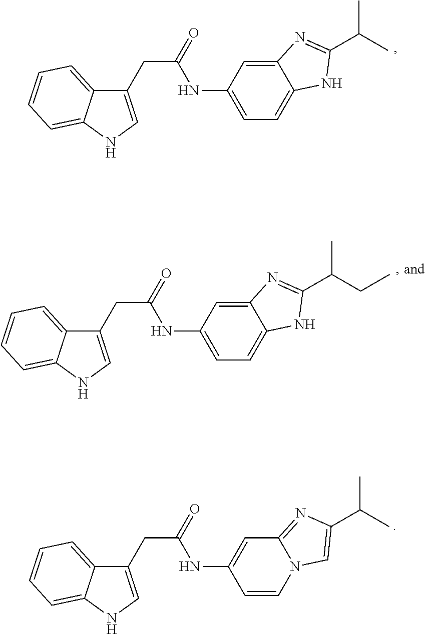 [SMILES: CC(C)c1nc2cc(NC(=O)Cc3c[nH]c4ccccc34)ccc2[nH]1 ; CCC(C)c1nc2cc(NC(=O)Cc3c[nH]c4ccccc34)ccc2[nH]1 ; CC(C)c1cn2ccc(NC(=O)Cc3c[nH]c4ccccc34)cc2n1]

Embodiment 6. The compound of embodiment 1, wherein the compound is

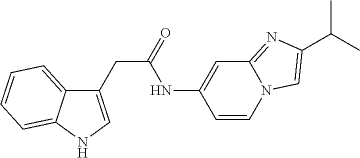 [SMILES: CC(C)c1cn2ccc(NC(=O)Cc3c[nH]c4ccccc34)cc2n1]

Embodiment 7. A pharmaceutical composition comprising the compound of any one of embodiments 1-6 and a pharmaceutically suitable carrier, diluent, or excipient.

Embodiment 8. A method for treating and/or preventing a disease or disorder associated with kalirin activity in a subject in need thereof, the method comprising administering to the subject the pharmaceutical composition of embodiment 7.

Embodiment 9. The method of embodiment 8, wherein the disease or disorder is neuropathic pain.

Embodiment 10. The method of embodiment 9, wherein the neuropathic pain is selected from the group consisting of post herpetic neuralgia, reflex sympathetic dystrophy, cancer pain, phantom limb pain, entrapment neuropathy, peripheral neuropathy, trigeminal neuralgia, and any combinations thereof.

Embodiment 11. The method of embodiment 8, wherein the disease or disorder is chronic pain.

Embodiment 12. The method of embodiment 11, wherein the chronic pain is selected from the group consisting of headache, nerve damage pain, low back pain, arthritis pain, fibromyalgia pain, and any combinations thereof.

Embodiment 13. The method of embodiment 8, wherein the disease or disorder is epilepsy.

### EXAMPLES

The following Examples are illustrative and should not be interpreted to limit the scope of the claimed subject matter. The following non-limiting Examples and data illustrate various aspects and features relating to the disclosed compounds, compositions, and methods including the treatment of diseases, disorders, and conditions associated with kalirin activity. While the utility of this invention is illustrated through the use of several compounds and compositions which can be used therewith, it will be understood by those skilled in the art that comparable results are obtainable with various other compound(s), as are commensurate with the scope of this invention.

### Example 1—Novel Kalirin Inhibitors for Regulation of Synaptic Plasticity and Neuropathic Pain Treatment

### Kalirin-A Master Regulator of Synapse Formation and Plasticity

Kalirin is a guanine exchange factor that activates the RAC-PAK pathway (Parnell et al, 2020). In response to NMDAr stimulation, Kalirin becomes activated. RAC1 activation induces AMPAr/NMDAr trafficking, synapse formation and dendritic spine growth. An emerging role has been isolated for Kalirin in regulating synaptic plasticity in the spinal dorsal horn (SDH) RAC inhibition and kalirin knockdown reduces neuropathic pain in animal models (Lu et al, 2014; Wang et al, 2016) by reducing synaptic plasticity in response to painful stimuli.

As such, a kalirin inhibitor would allow tissue and pathway selective inhibition of neuropathic pain.

### Kalirin—A Route to Regulate RAC Signaling in Neurons to Regulate Synaptic Plasticity and Connectivity

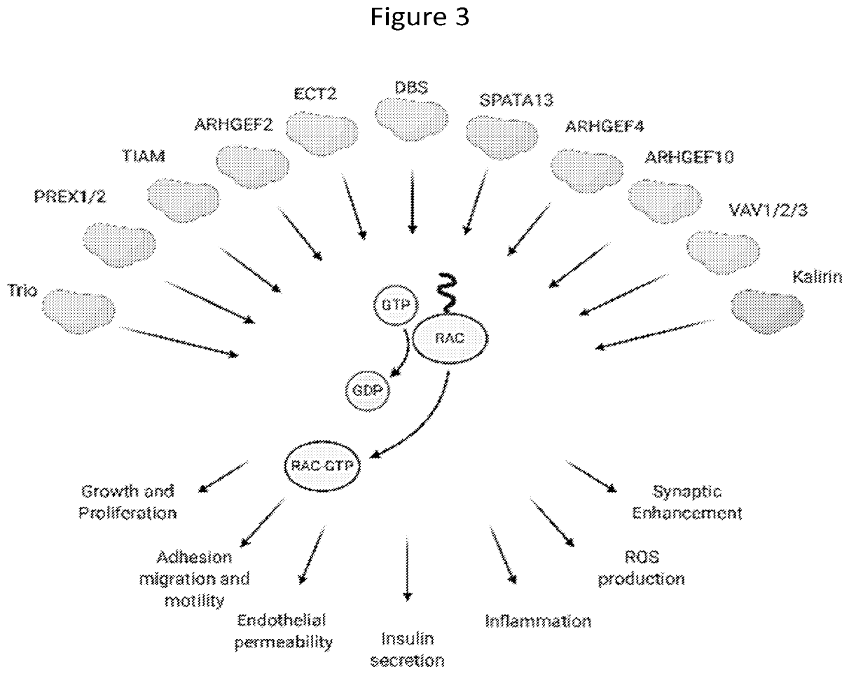

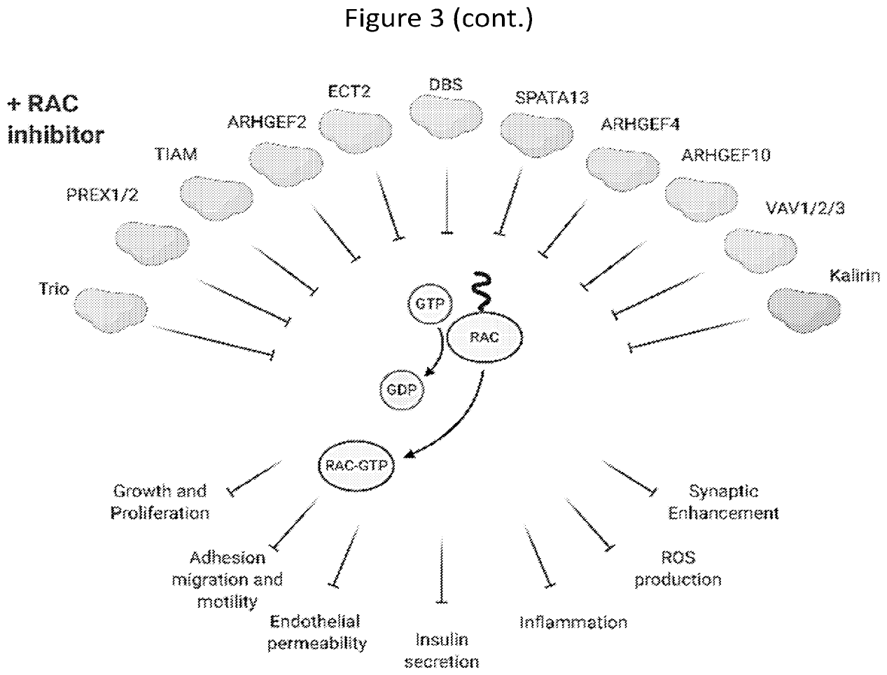

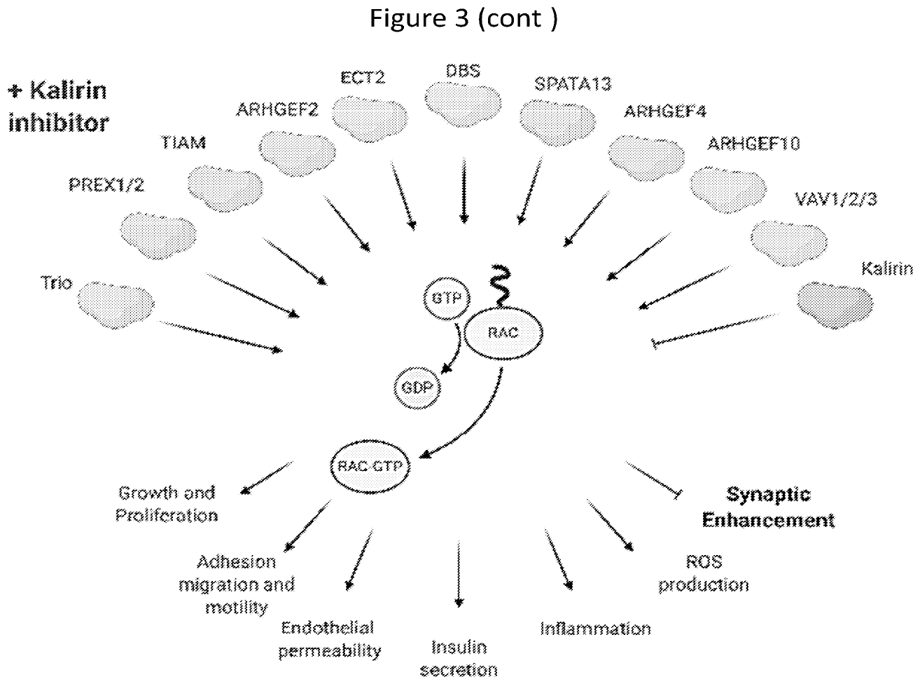

RAC1 is a key regulator of numerous processes (FIG. 3). Inhibition of RAC1 has shown efficacy in culture models to regulate many of these processes. The specific downstream effects are induced by a host of upstream GEFs that activate RAC1 in tissue specific manners, and in a spatially restricted manner within the cells (FIG. 3). A number of non-specific RAC inhibitors allow inhibition of these process in vitro. However, due to widespread expression of RAC1, these compounds have not progressed to preclinical/clinical assessment due to off-target inhibition and poor pharmokinetic properties. Inhibition of specific upstream GEFs may allow tissue and subcellular control of RAC1 signaling.

As such, kalirin inhibition provides a route to specifically inhibit synaptic activity/structure.

Kalirin—A Functionally/Spatially Restricted GEF with Precise Control Over Synaptic Function

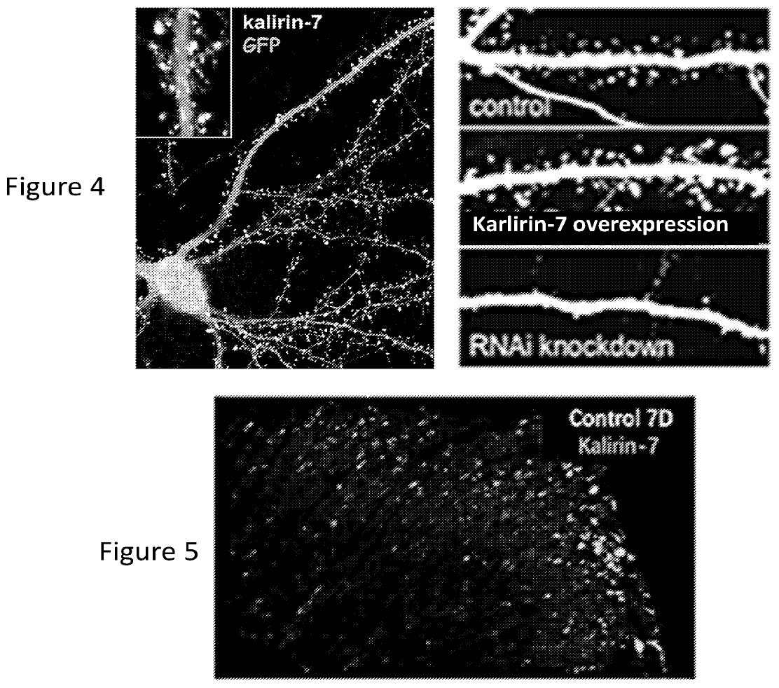

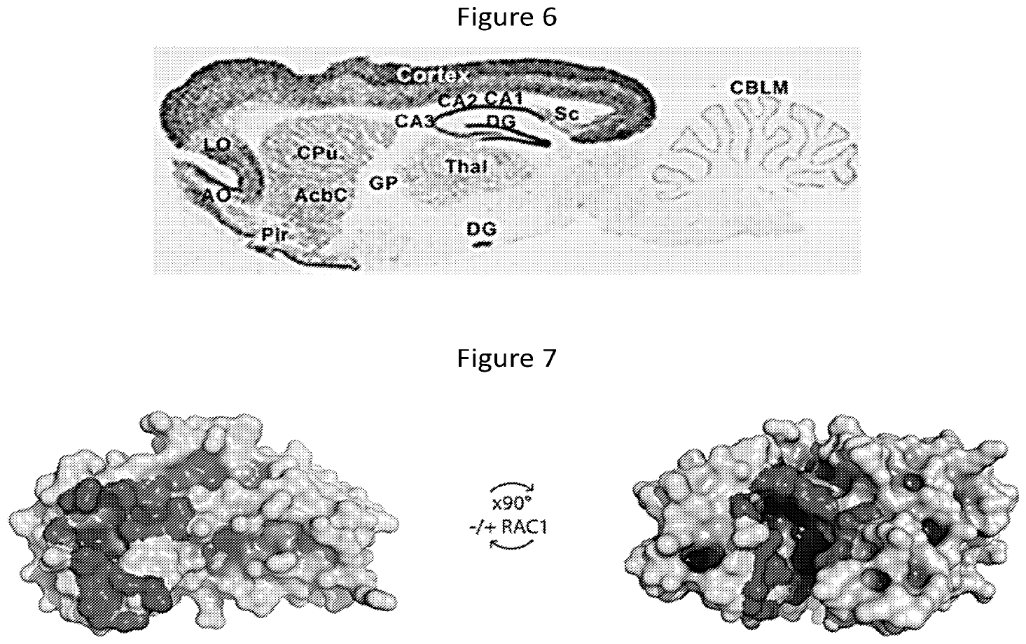

Kalirin provides an exceptional target for specific regulation of RAC1 activity in neurons and synapses, due to its restricted expression within dendritic spines (FIG. 4) and the spinal cord and CNS (FIG. 5, taken from Lu et al, 2020; FIG. 6). Within dendritic spines, kalirin promotes the formation and growth of dendritic spines (FIG. 6). Knockdown of kalirin in animal models has proven effective at inhibiting pain sensitization.

As such, an inhibitor of kalirin may have utility in reducing neuropathic pain by blocking synaptic plasticity in the spinal dorsal horn.

### Defining a Novel Selective Interfacial Pocket in the Kalirin/RAC Interface

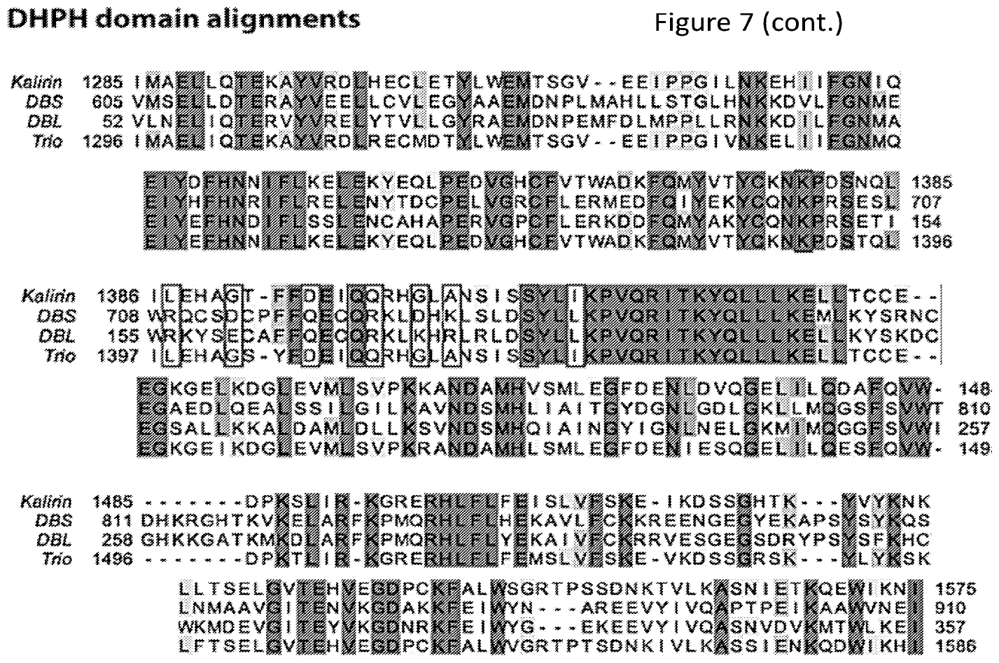

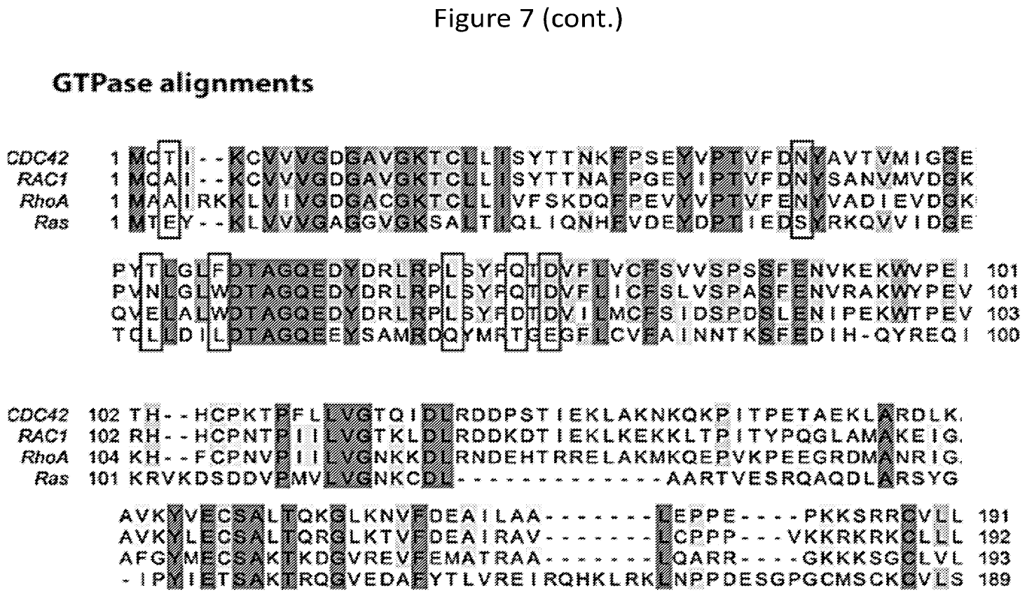

Kalirin catalyzes GDP release from RAC1 via its N-terminal DHPH guanine exchange factor (GEF) cassette (FIG. 7). We have identified a pocket formed between kalirin and RAC1 during kalirin mediated GEF catalytic activity (FIG. 7). This site provides a binding pocket suitable for drug binding and composed of surface exposed residues showing low homology to related GEF proteins. In silico screening of crystal structure 5033 of the kalirin-RAC1 interface was performed to identify compounds predicted to bind to this site.

As such, this site represents a novel druggable target, suitable for drug discovery efforts.

### In Silico Screening Reveals a Small Molecule Inhibitor of Kalirin

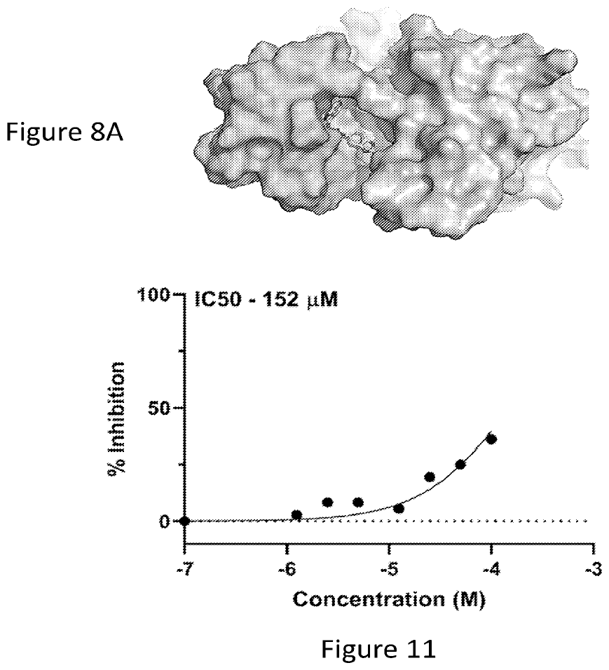

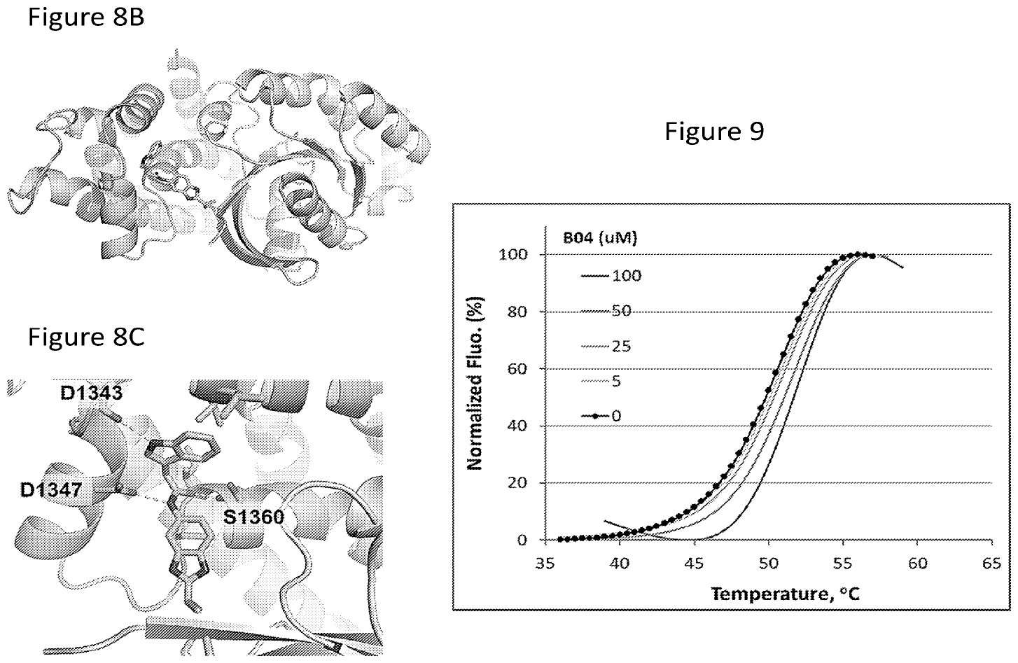

Compound PP01 (i.e. B04 or NW01) was predicted to bind to the interfacial Kalirin-RAC1 interface (FIGS. 8A-8C). Hydrogen bonding is predicted to occur between B04 and kalirin (residues D1342, D1347, S1360). Compound B04 may act to stabilize (uncompetitive) or disrupt (competitive) kalirin-RAC1 interaction.

Isolated compound B04 is predicted to bind at a novel, uncharacterized binding site.

B04 is shown to promote the thermodynamic stability of the kalirin GEF domain, indicative of direct interaction by fluorescence thermal shift (FTS). B04 inhibits kalirin biochemical activity but has no effect on the intrinsic release of bGDP from RAC1. This was assessed using a miniaturized high-throughput GEF assay, utilizing fluorescently labelled GDP (bodipy-GDP) loaded RAC1. B04 inhibits with a mid micromolar affinity (similar to previously isolated RAC-inhibitory compounds) (FIGS. 8A-8C and FIG. 9).

As such, B04 inhibits biochemical activity of kalirin-GEF activity.

### Isolated Compound Shows Favorable Pharmokinetic Properties

B04 was assessed based on predicted pharmokinetic profile using SwissADME (Daina et al, 2016): it is Lipinski and Veber compliant; pan-assay interference agents negative; and predicted GI and blood-brain barrier permeable.

These predictions suggest suitability of B04 for in vivo applications.

### Isolated Compound Shows Low Toxicity and Cellular Efficacy

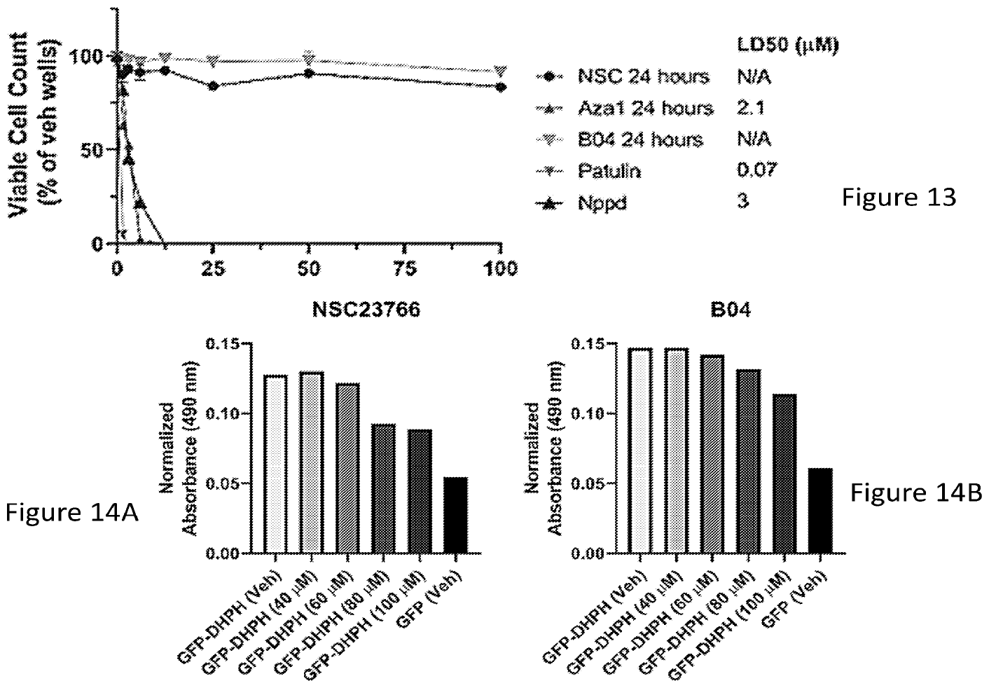

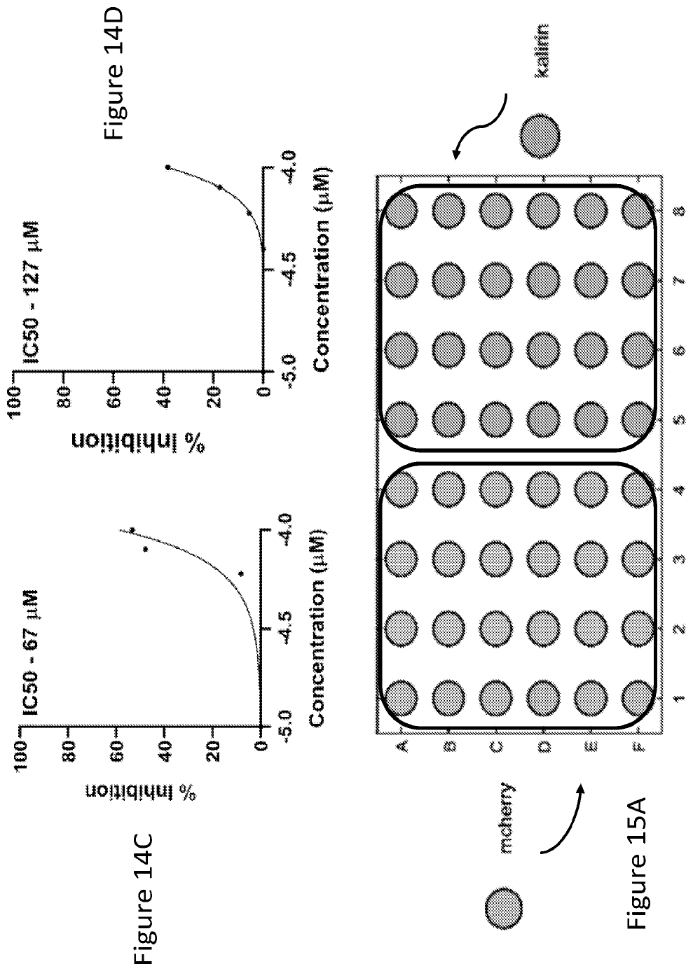

Compound was incubated with heterologous cells (HEK293T) for 24 hours, and cell death was measured using the Cell-titre Glo assay. Previously published GTPase inhibitor compounds NSC23766 (NSC), AZA1, patulin and NPPD were tested alongside B04. Only NSC23766 and B04 were well tolerated, suggesting suitability for cellular and neuronal assays (FIG. 13). Cells were transfected with the kalirin-GEF domain, and assessed for RAC1 activation using the RAC-Glisa assay (Cytoskeleton org.) B04 (FIGS. 14B and 14D) and NSC23766 (FIGS. 14A and 14C) were found to inhibit kalirin activity in line with published (Gao et al 2004) IC50s and biochemical analysis (FIG. 14A-14D).

B04 shows no toxicity and is cell permeable. These data support B04 in cellular inhibition of kalirin.

### Expected Effects of Kalirin on MEA Data

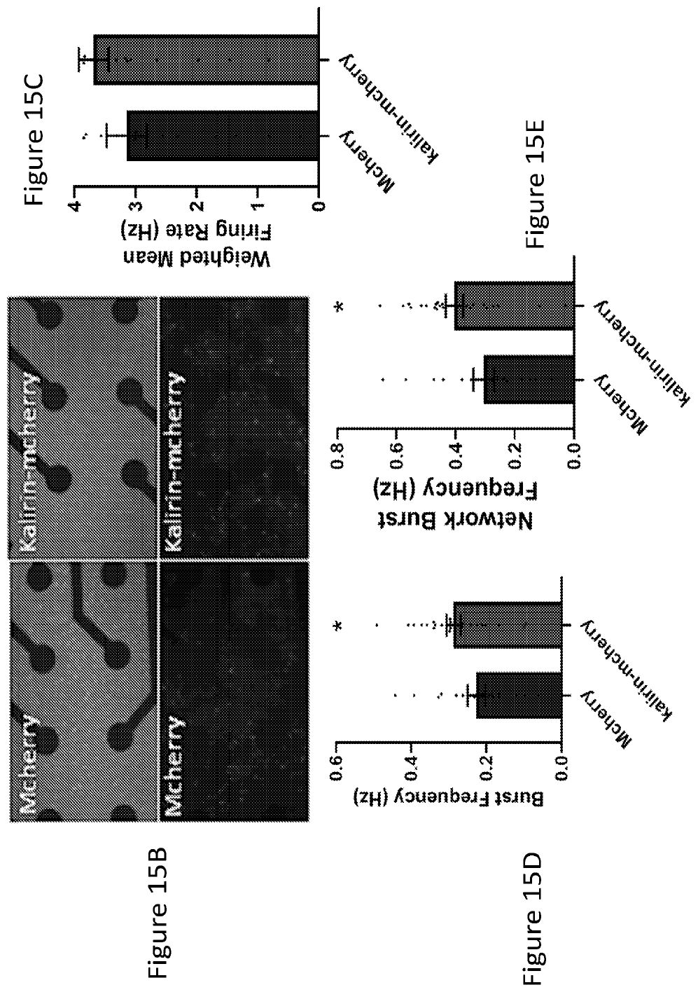

Rat neurons were infected with kalirin-overexpression virus (synapsin promoter driven) alongside synapsin-mcherry control virus, at 14 DIV (FIG. 15A-15B). Expression for 7 DIV, before reading activity. Kalirin was found to significantly increase burst firing and network firing and has no effect on mean firing rate (FIG. 15C-15E).

Kalirin overexpression is found to alter burst frequency and network burst frequency in neuronal lines.

### Neuronal Efficacy in Reducing Electrical Activity

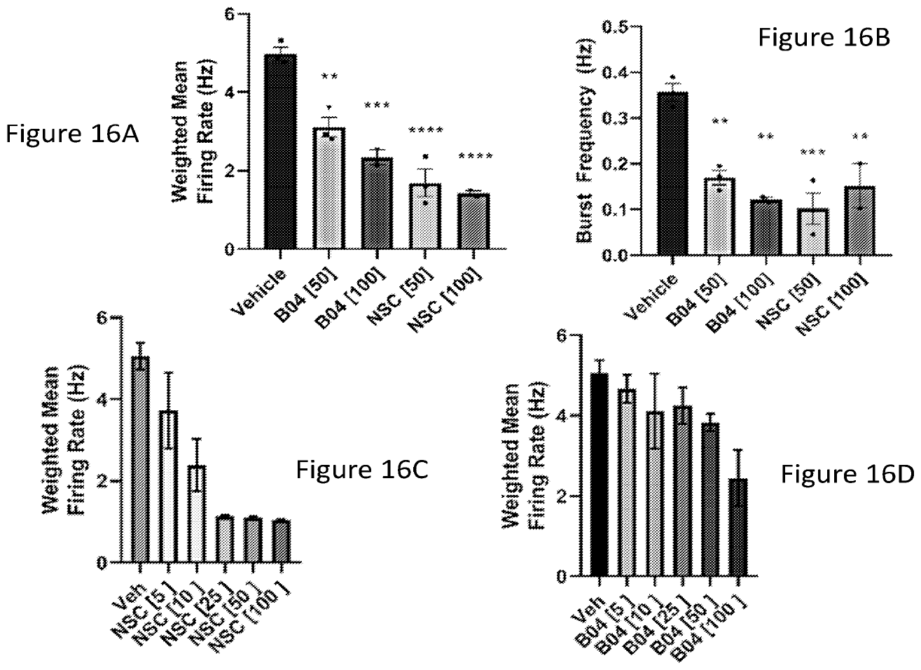

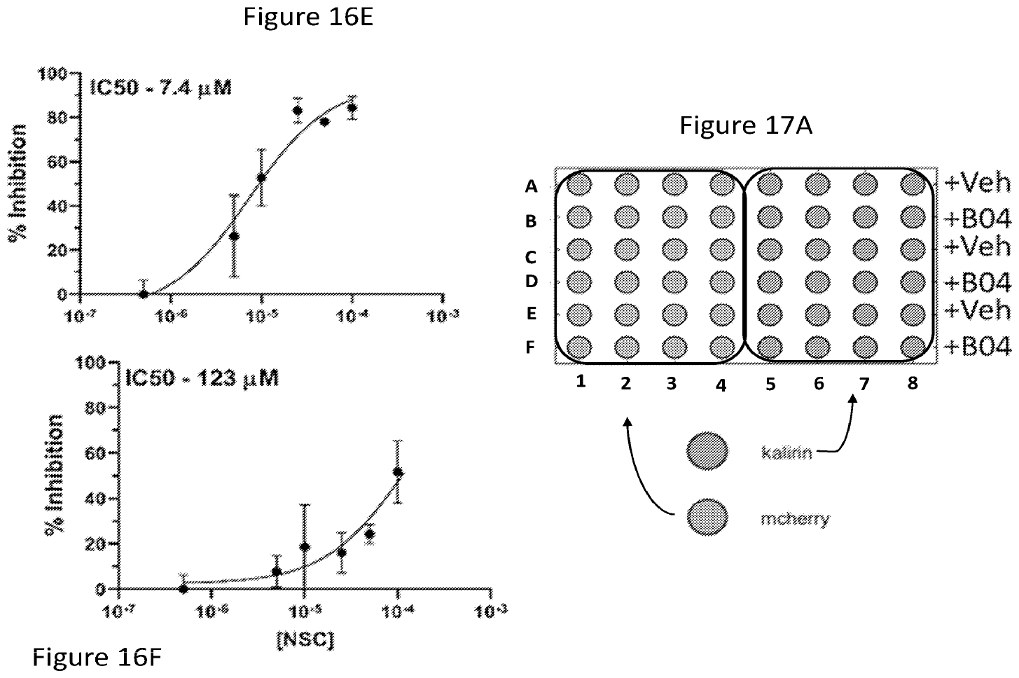

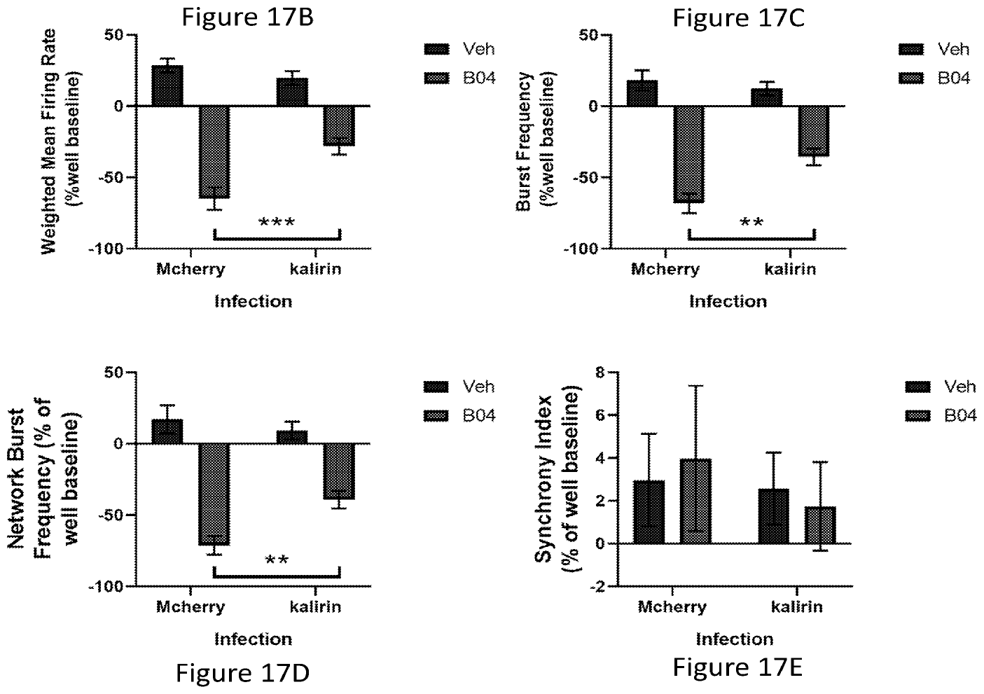

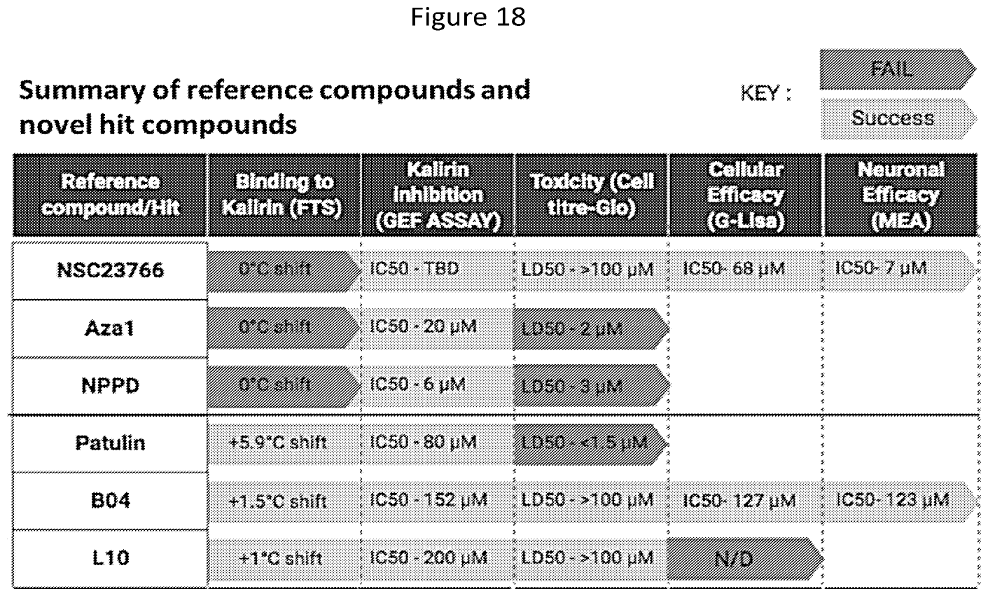

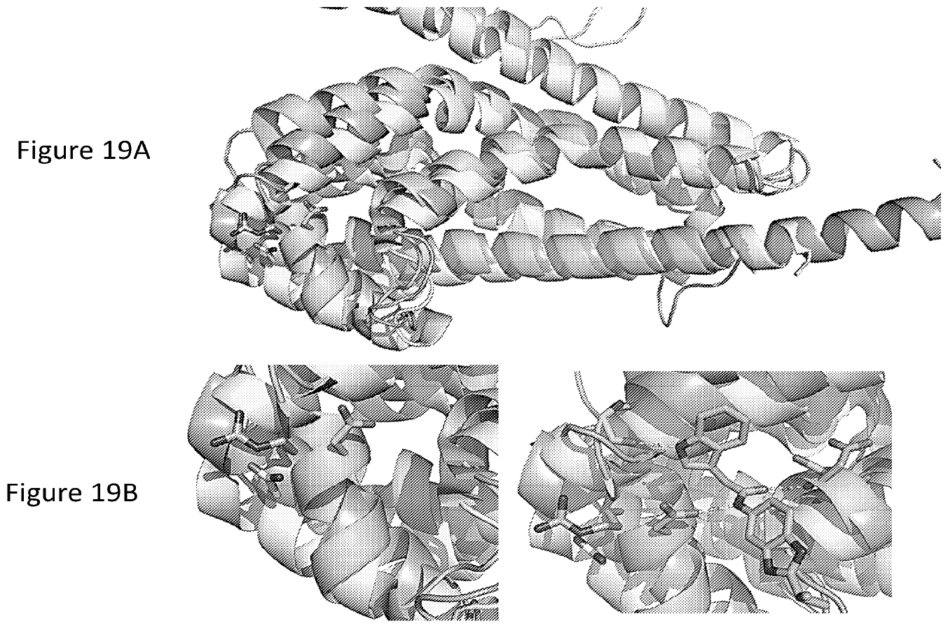

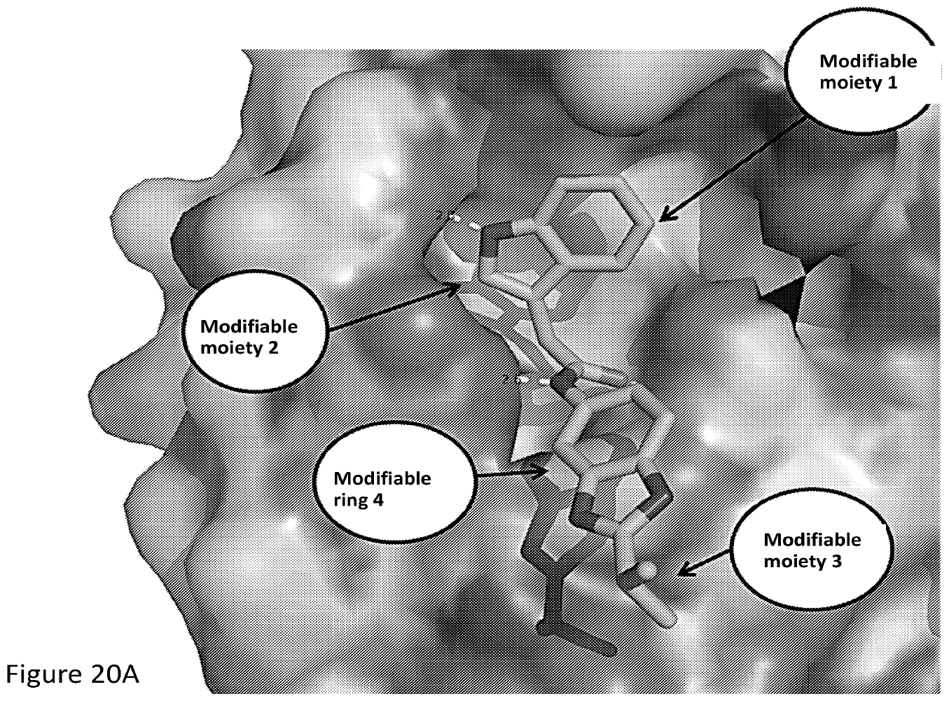

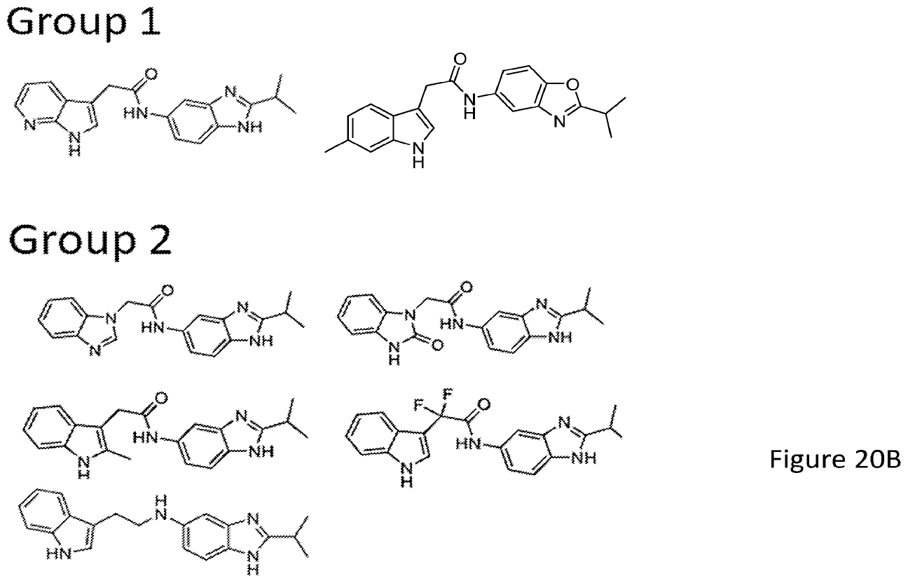

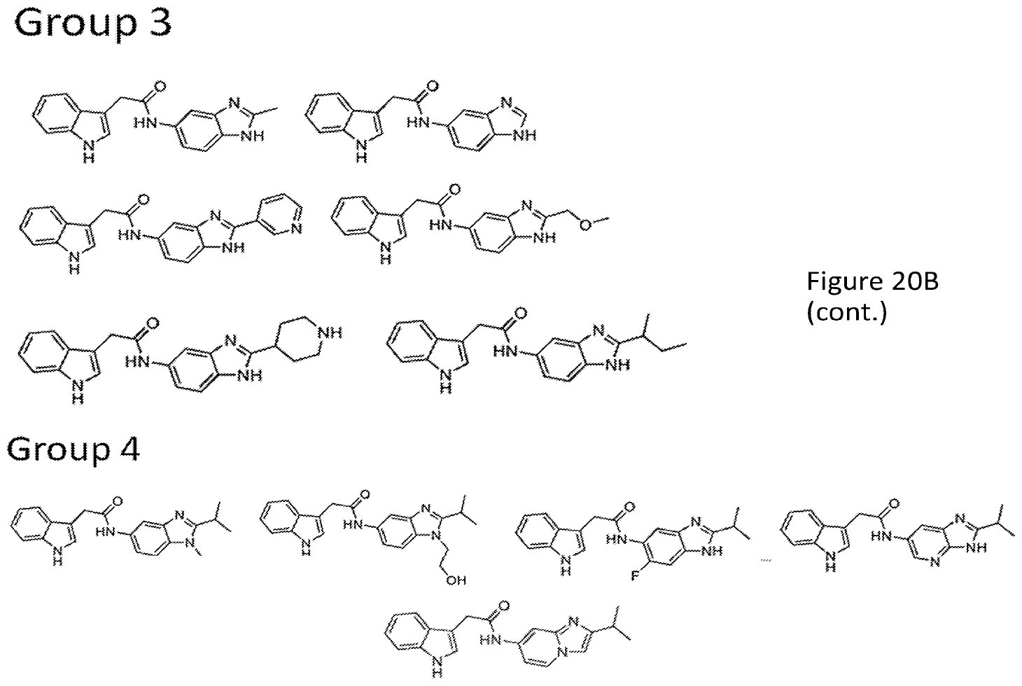

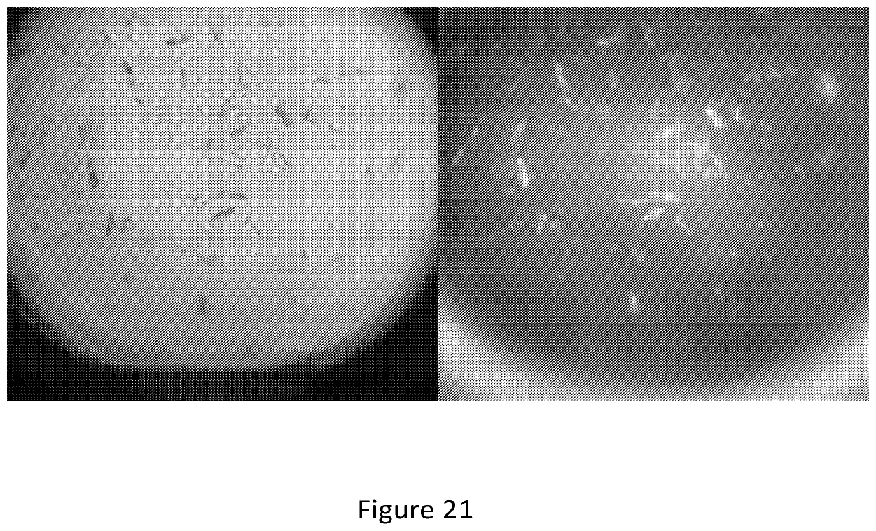

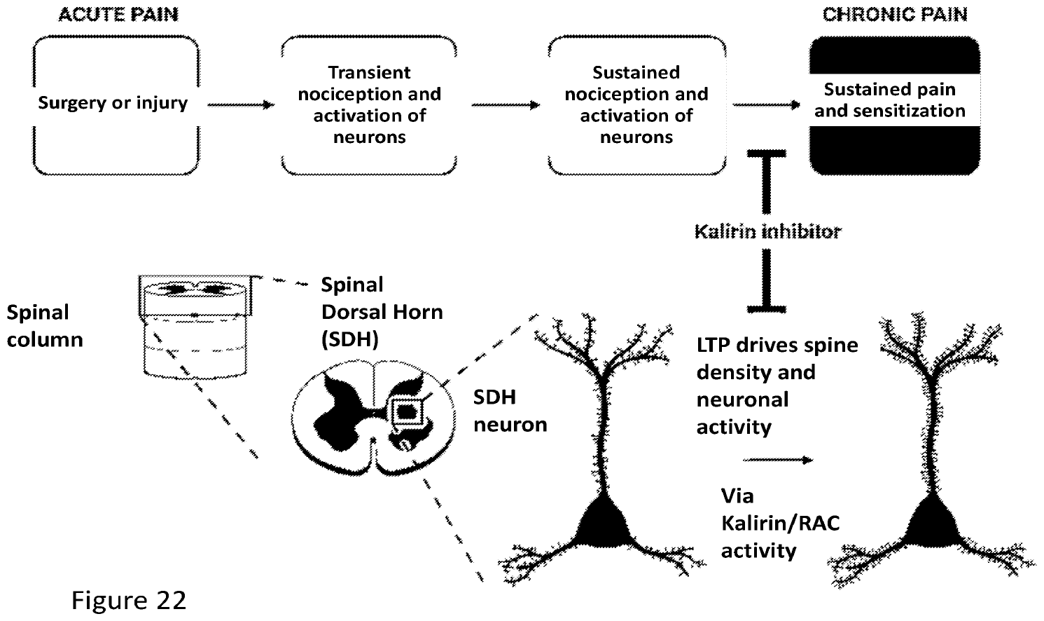

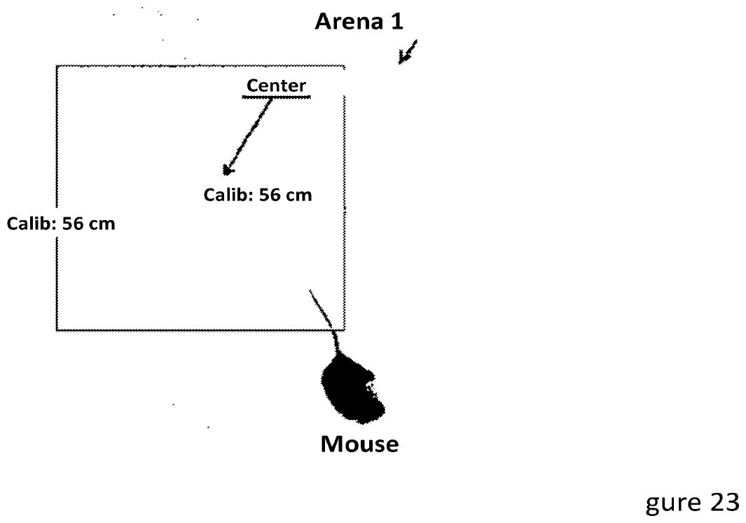

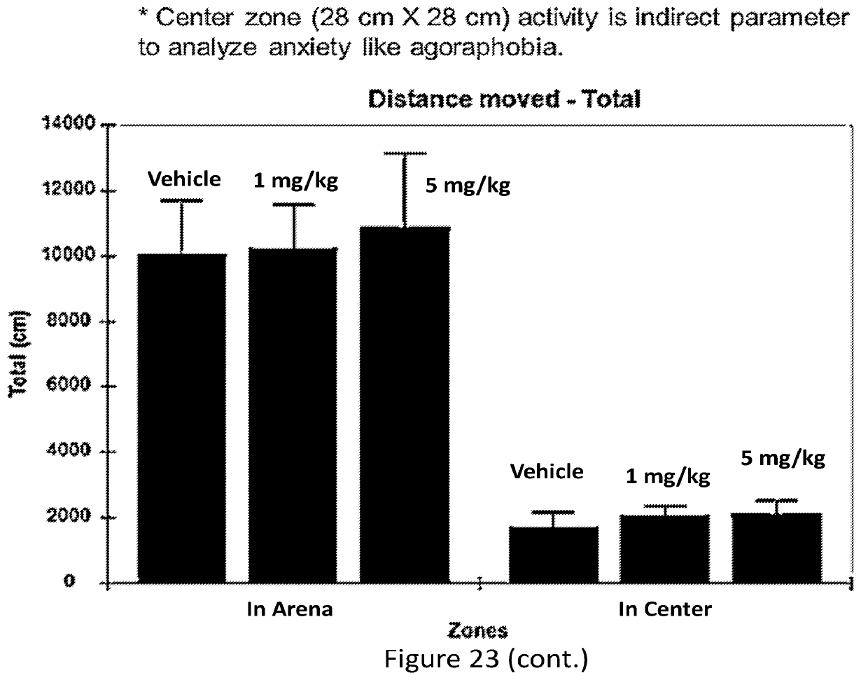

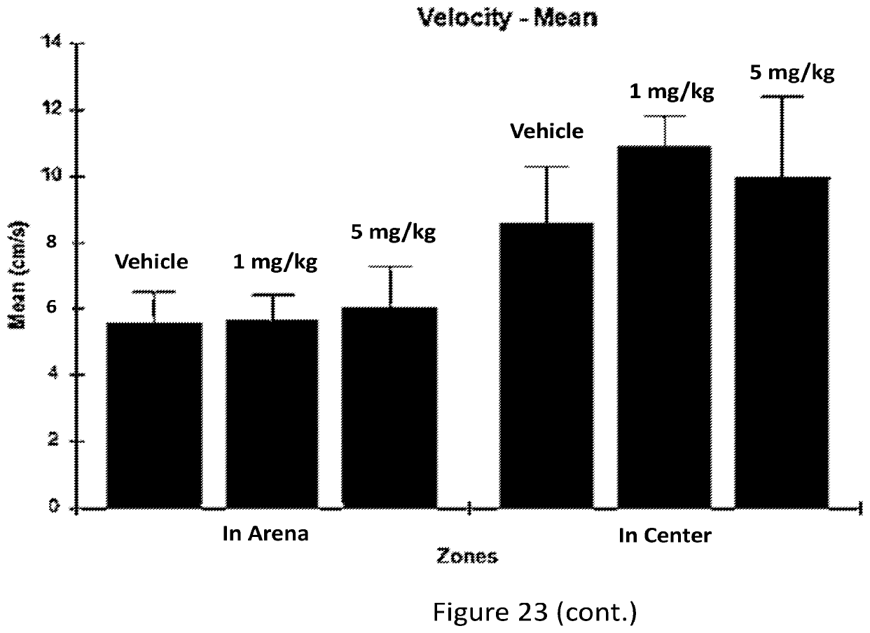

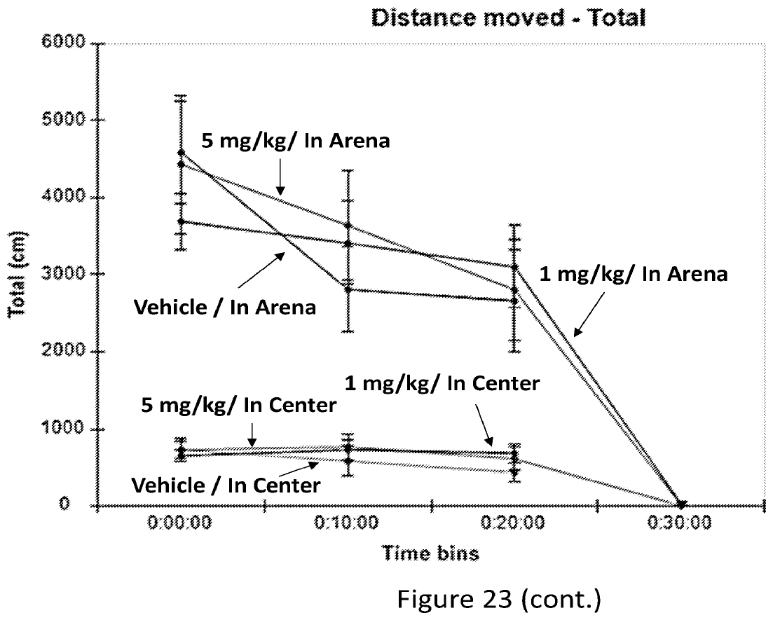

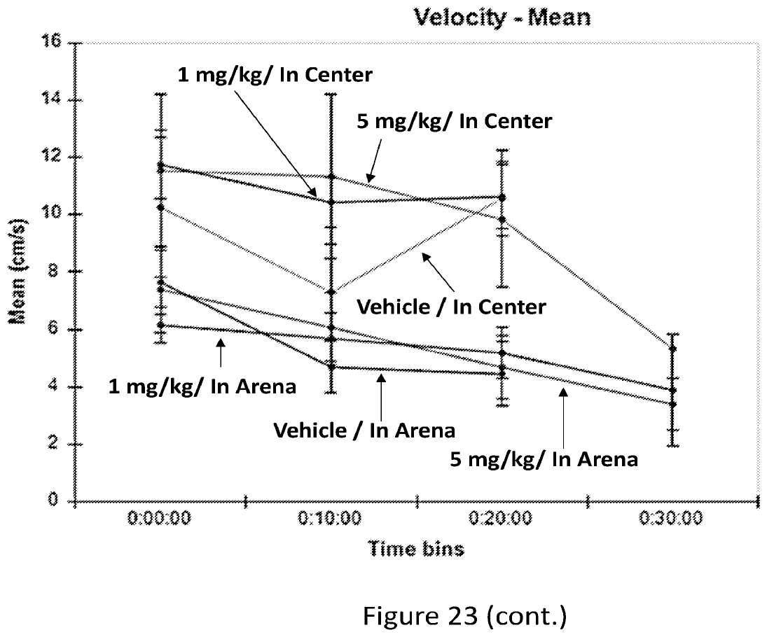

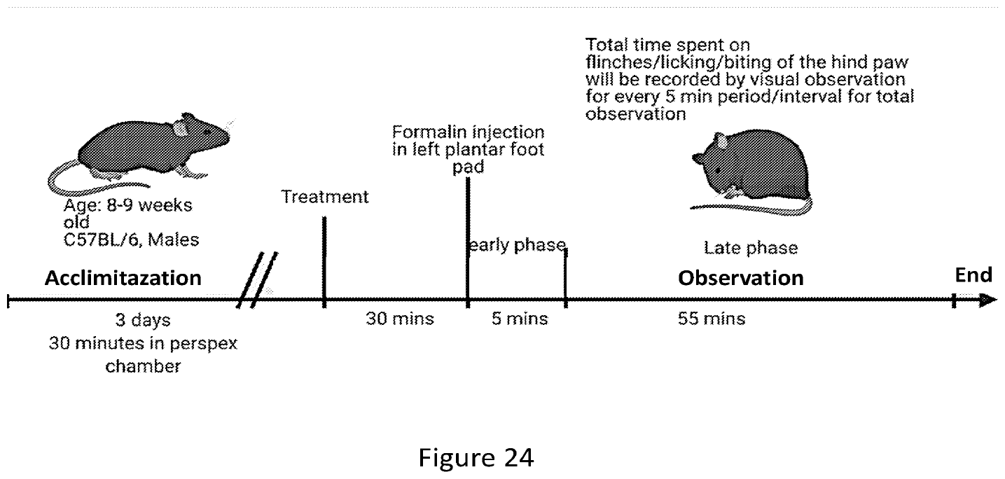

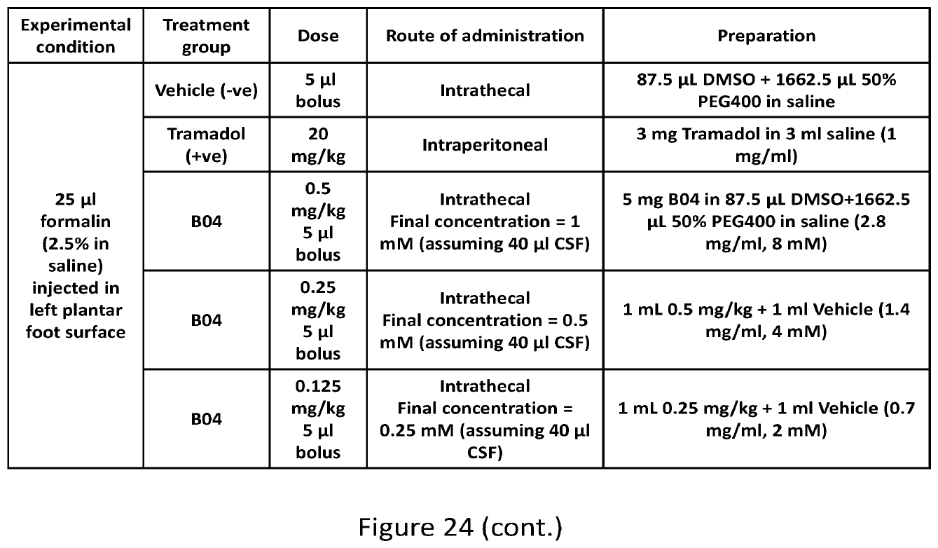

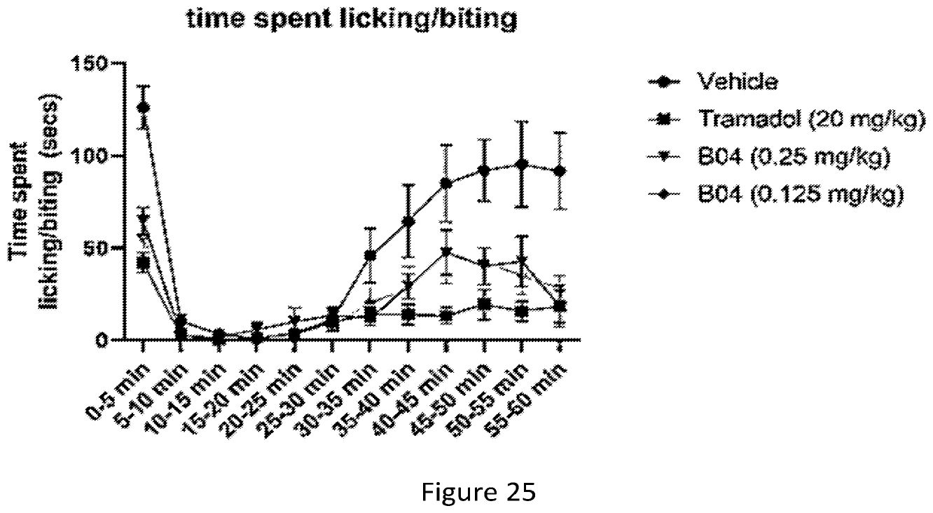

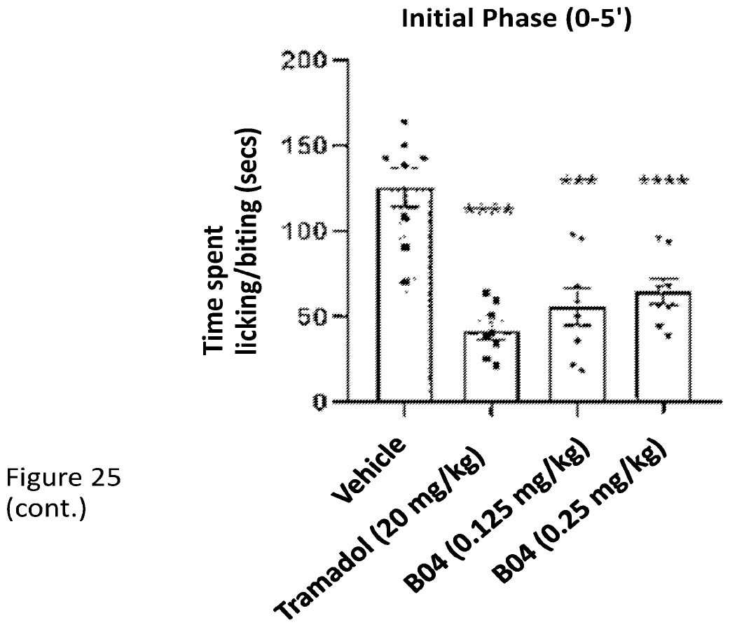

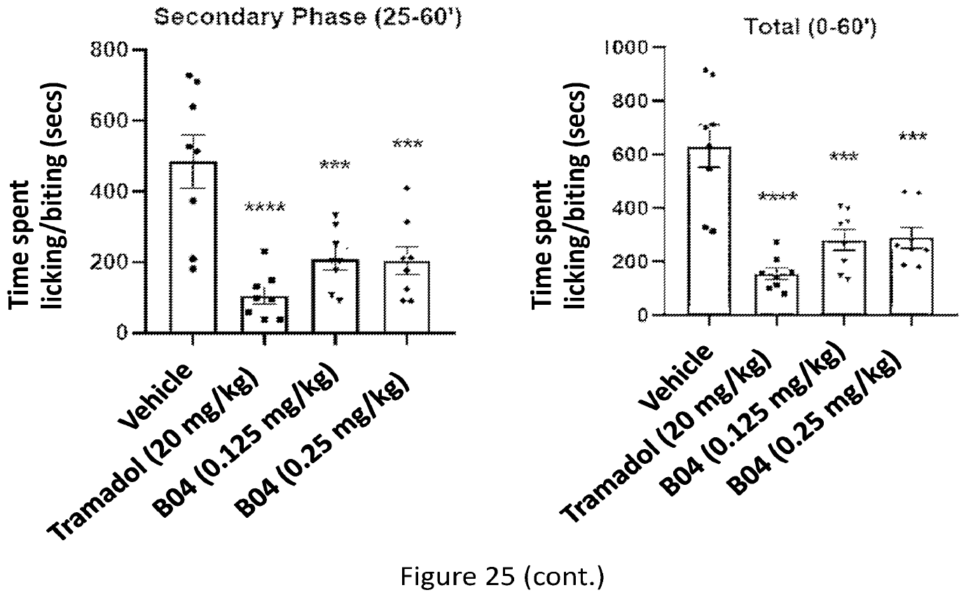

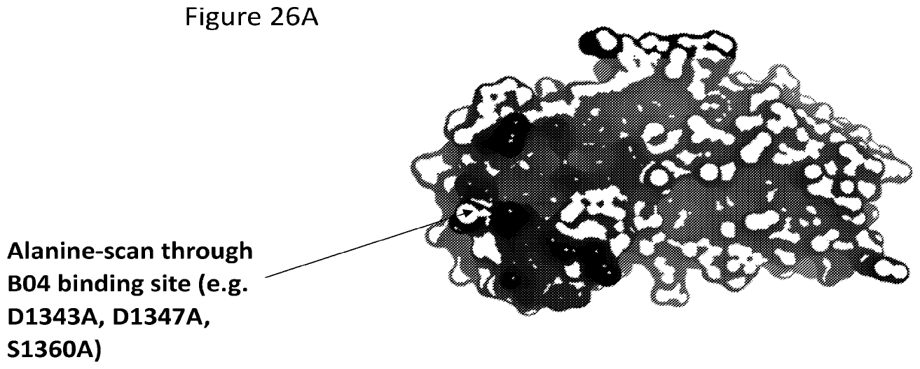

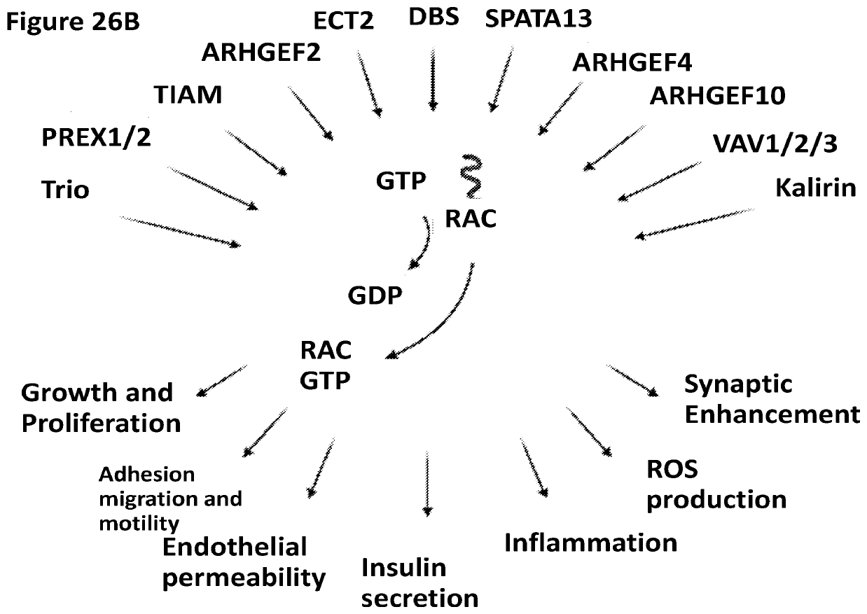

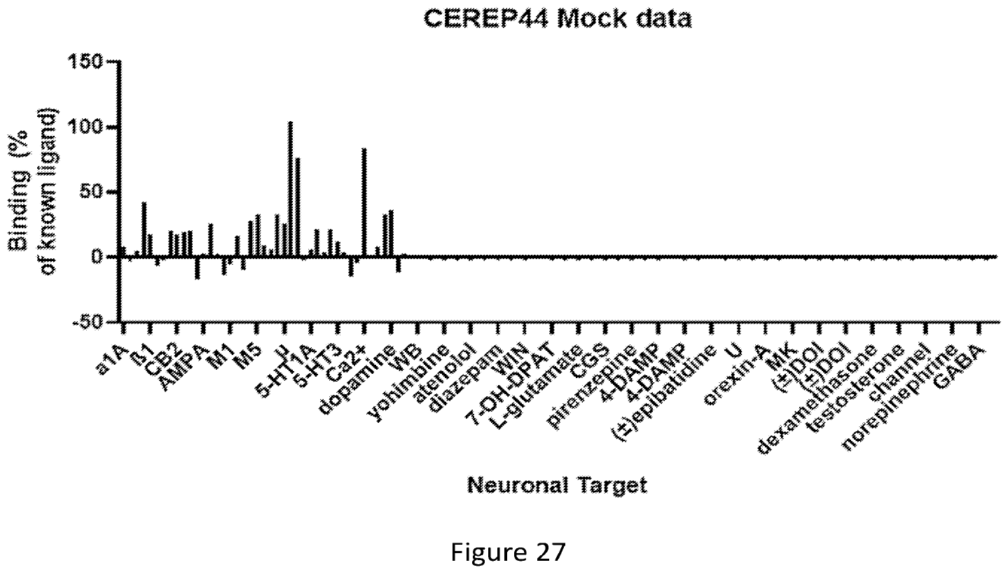

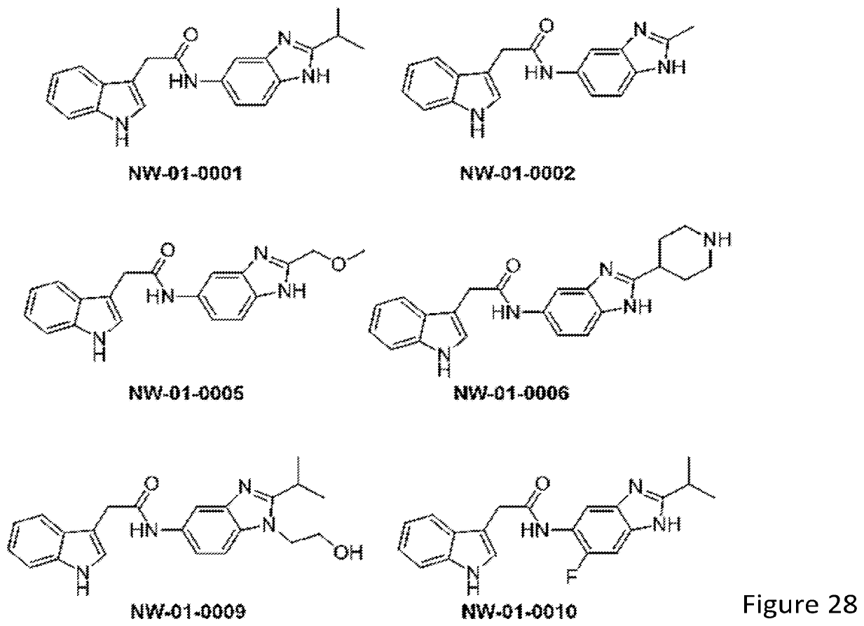

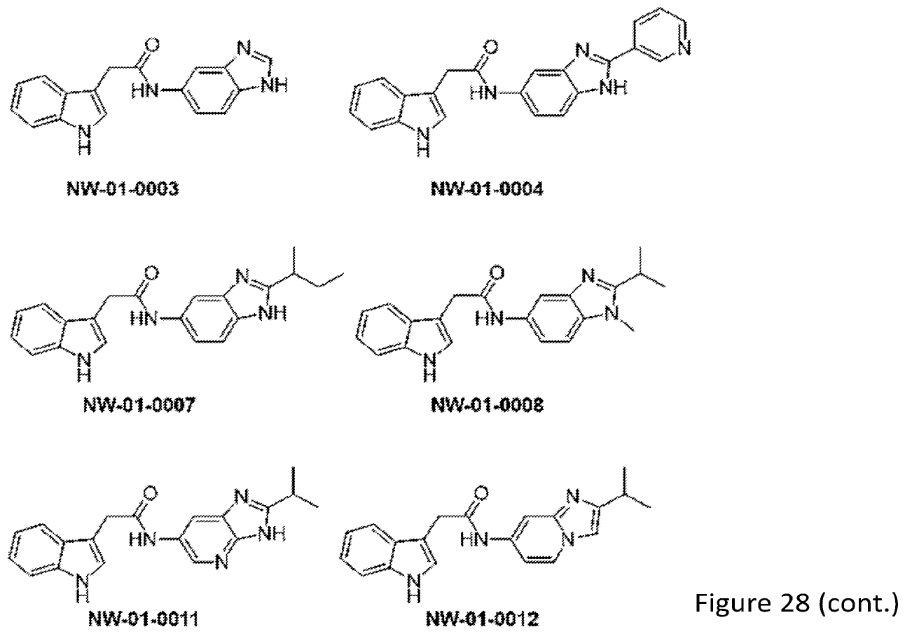

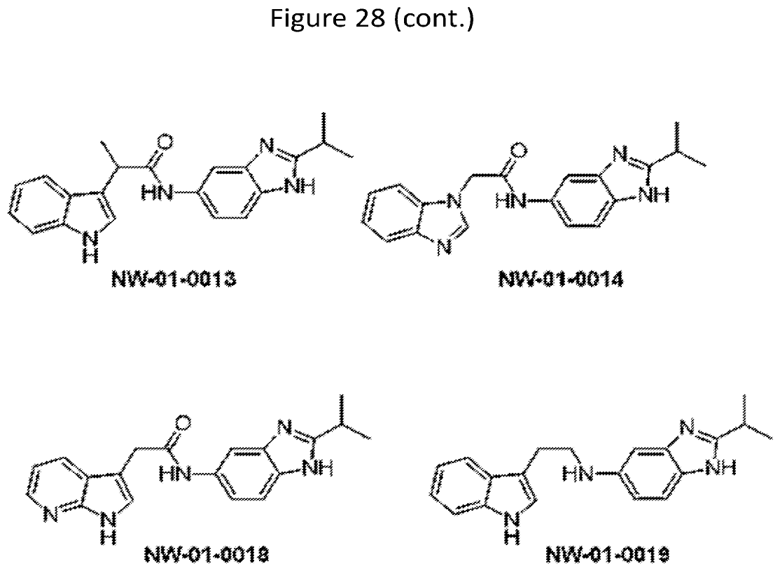

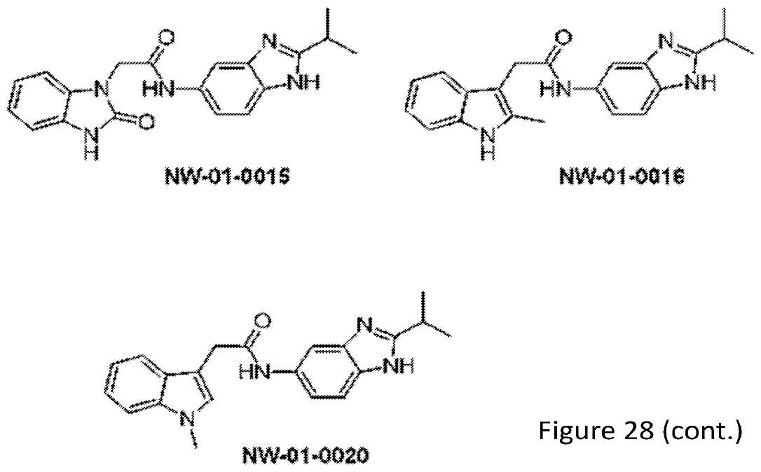

48 well multi-electrode arrays (MEAs) were seeded with 50,000 primary cultured rat neurons and grown to 3WIV. Cells were incubated with NSC23766 or B04 at 50 and 100 μM for 4 hours alongside vehicle control (1% DMSO, FIG. 16A-16B). Neuronal activity was assessed in terms of weighted mean firing rate (number of events per second across active electrodes) and burst frequency (>5 events in 100 ms within a single electrode) and network burst frequency (FIG. 16A-16D).

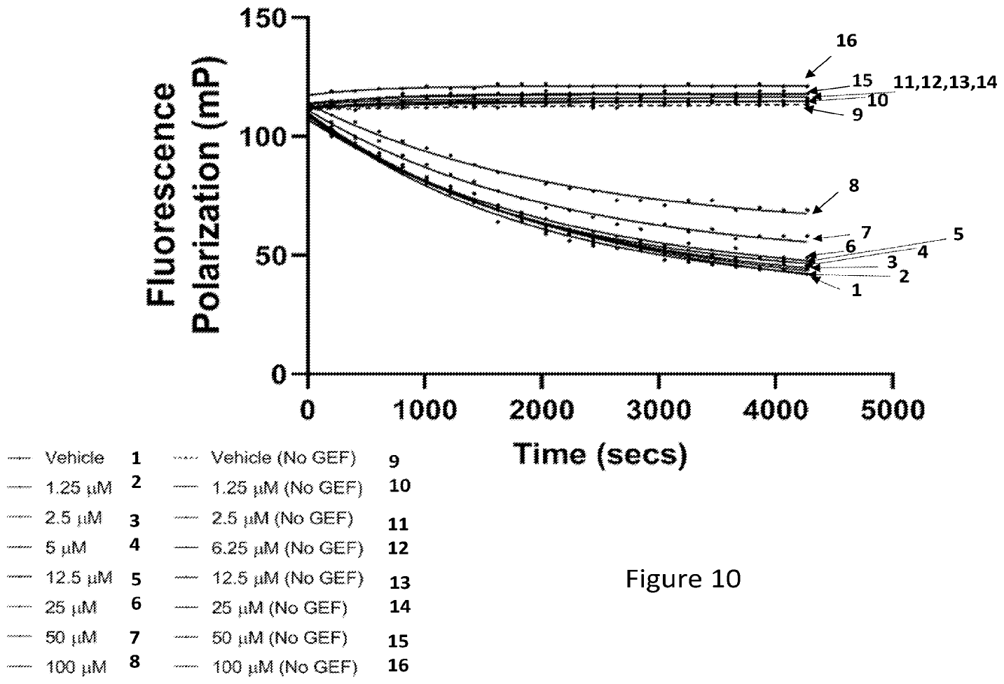

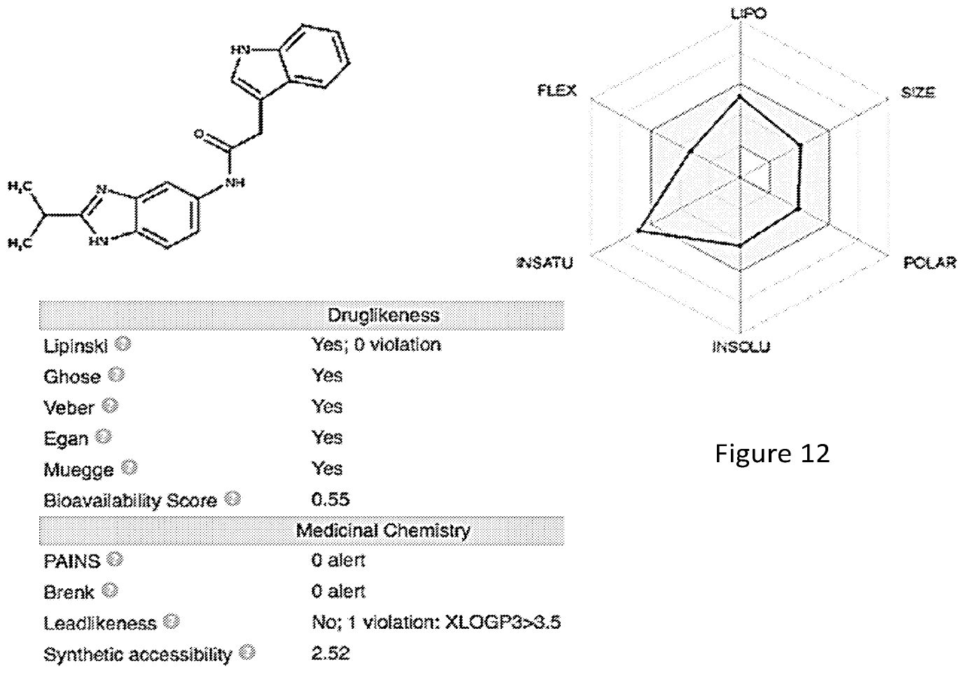

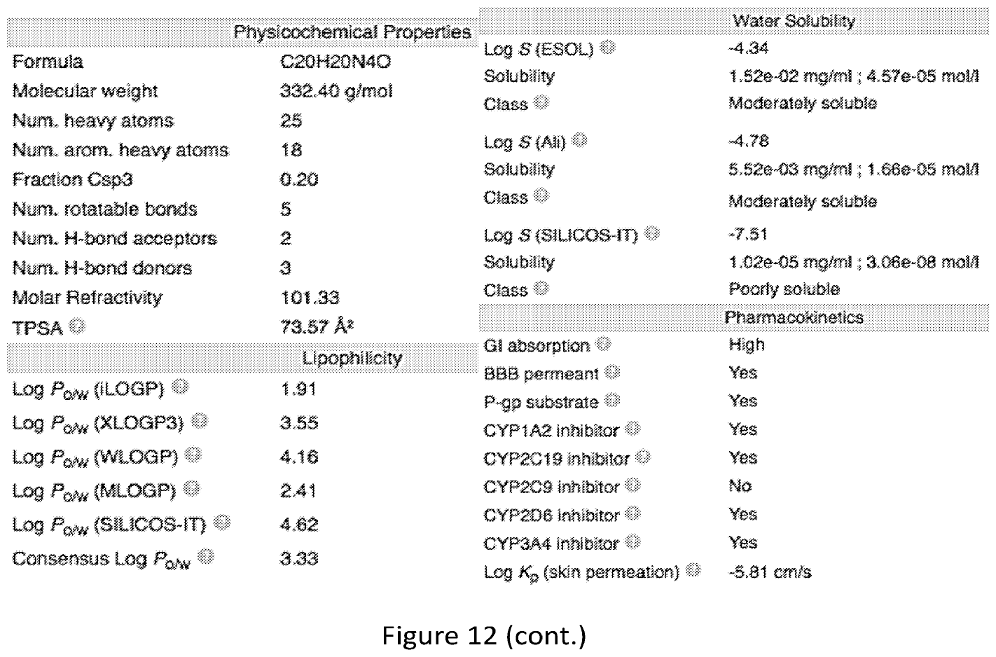

DRC curve analyses of NS23766 (FIGS. 16C and 16E) and B04 (FIGS. 16D and 16F) was performed to assess compound efficacy. NSC23766 was found to have a 10× greater inhibitory efficacy compared to published reports (Gao et al, 2004) and cellular activity (FIGS. 15C and 15E). This is likely due to reported off-target inhibition of NMDAr (Hou et al 2014). B04 was found to inhibit electrical activity similarly to biochemical (FIG. 10 and FIG. 11) and heterologous values (FIGS. 14B and 14D).

Interestingly, B04 produced a significant decrease in WMFR, despite overexpression failing to induce significant alterations in firing rate. This may be due to the chronic vs transient nature of kalirin regulation, highlighting the need for tools to assess kalirin activity acutely.

These data support the efficacy of B04 in reducing neuronal activity, consistent with kalirin inhibition.

### Target Engagement

Plate was treated with vehicle control or B04 for 4 hours. The % change from baseline was recorded. Significant decrease in B04 efficacy induced by kalirin overexpression (Firing rate, burst frequency and network burst frequency). Drug resistance from overexpression is a known phenomenon, supporting target engagement (Palmer et al, 2014).

These data suggest target engagement of B04 in selectively inhibiting kalirin activity.

### A Summary of B04 Assays

We tested known inhibitors of the RAC pathway in our suite of biochemical, heterologous and neuronal assays. Previously isolated RAC inhibitors (NSC23766 and AZA1, and a proposed kalirin inhibitor (NPPD) were found to not bind to kalirin by FTS, but inhibited GEF activity in line with published results.

B04, along with other in-house hit compounds (L10 and patulin) bond to kalirin and inhibited GEF activity. Only B04 and NSC23766 were suitable for neuronal studies due to toxicity. Whereas B04 efficacy was recapitulated in all assays (supporting target engagement), NSC23766 efficacy was 10× higher in neurons (suggesting off-target inhibition).

B04 outperforms existing RAC pathway inhibitors, and selectively inhibits neuronal activity.

### Drug Development

We have generated a custom library of related compounds. Compound library was generated by modifying moieties on B04 predicted to increase affinity. These may be employed in FTS, biochemical GEF assays, heterologous GEF assays and MEA neuronal assays to find higher efficacy compounds sharing the B04 backbone.

The predicted binding pocket shows suitability for selective GEF inhibition (related GEF proteins show low homology/structural similarity at this site.

Co-crystallization of kalirin-GEF domain may be performed with B04. This will confirm binding site, and allow assessment of structural reorganization that may alter secondary structure upon drug binding. This can go hand in hand with ligand observe NMR to assess binding to the GEF domain, and 2D protein-NMR to assess residues undergoing chemical shift upon B04 incubation. These will allow informed modification of B04 substructure to optimize binding and efficacy.

### In Vivo Efficacy

In order to validate the in vivo efficacy and disease relevance of B04, we have designed a suite of behavioral assays. These assays will assess the reduction of pain-induced synaptic plasticity within the spinal dorsal horn and concurrent cessation of neuropathic pain. PK studies to assess ADMET properties, bioavailability, toxicity and blood brain permeability. The following assays and experiments will be conducted: inflammatory pain cessation (formalin and CFA assay), neuropathic pain cessation (spinal nerve ligation assay), multi-electrode array (ex-vivo) of spinal cord tissue, C-Fos staining of spinal cord tissues to assess neuronal inhibition following treatment.

### Target Selectivity and Potential for Selective GEF Family Inhibitor Discovery

In order to validate the selectivity of kalirin, we can generate active GEF protein libraries suitable for assessment of off-target GEF inhibition.

### Site Directed Mutagenesis of Key B04 Interacting Residues

We may perform alanine scanning to confirm the proposed binding site identified from in silico screening. Ala-mutants will be assessed for catalytic activity and folding. Ala-mutants will be tested alongside WT kalirin to assess binding (by FTS/ligand observe NMR) and compound efficacy (GEF assay).

### In-House GEF Protein Library

Related GEFs show the highest structural and sequence homology to kalirin within the proposed drug binding site. We can generate a library of GEF domains, validated for activity. B04 will be assessed in terms of selectivity against this library.

### Example 2—Novel Kalirin Inhibitors with High Efficacy

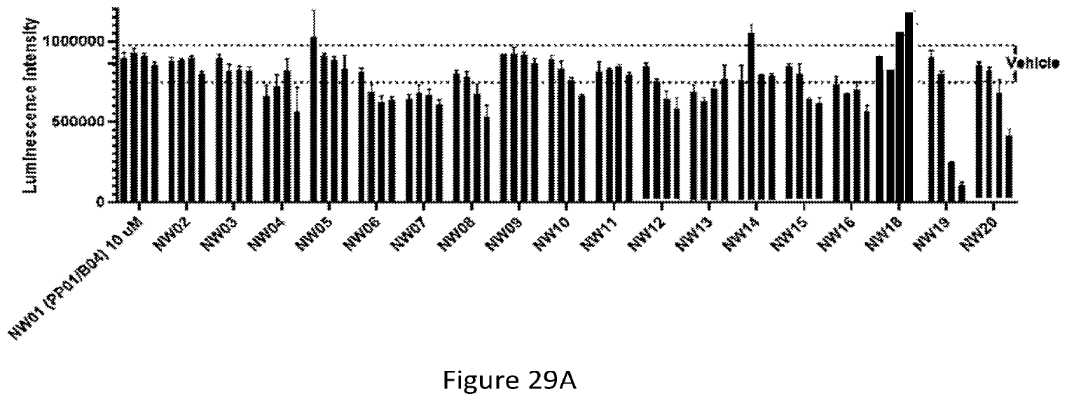

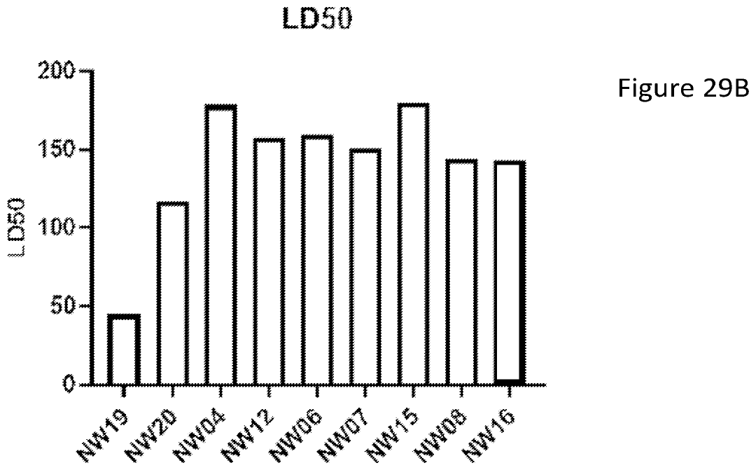

FIGS. 29A and 29B show the toxicity of PP01 analogues. The syngene hits and LD50 data of selected compounds show that compound PP01B (i.e. NW12) is suitable for in vitro cellular studies.

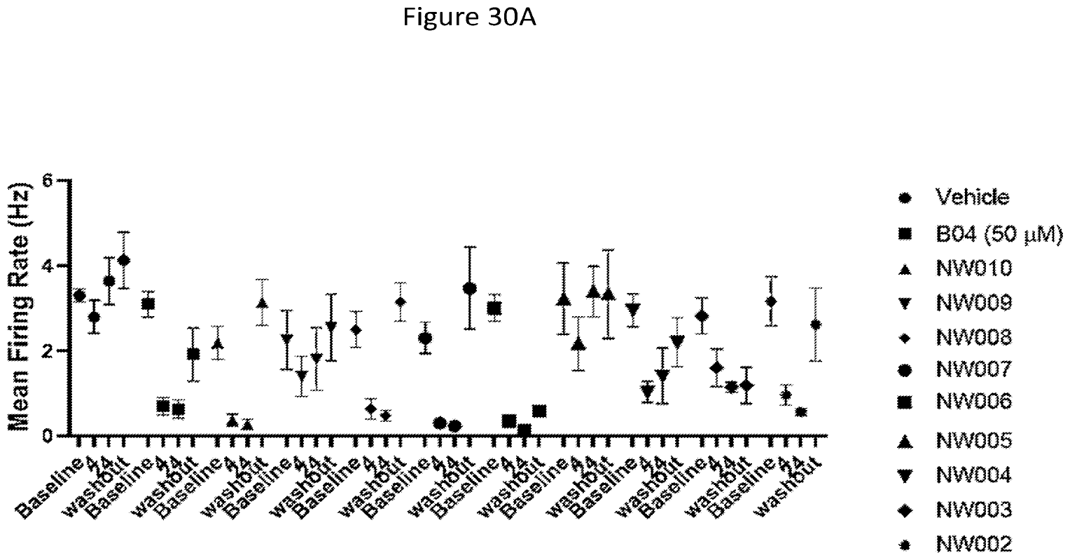

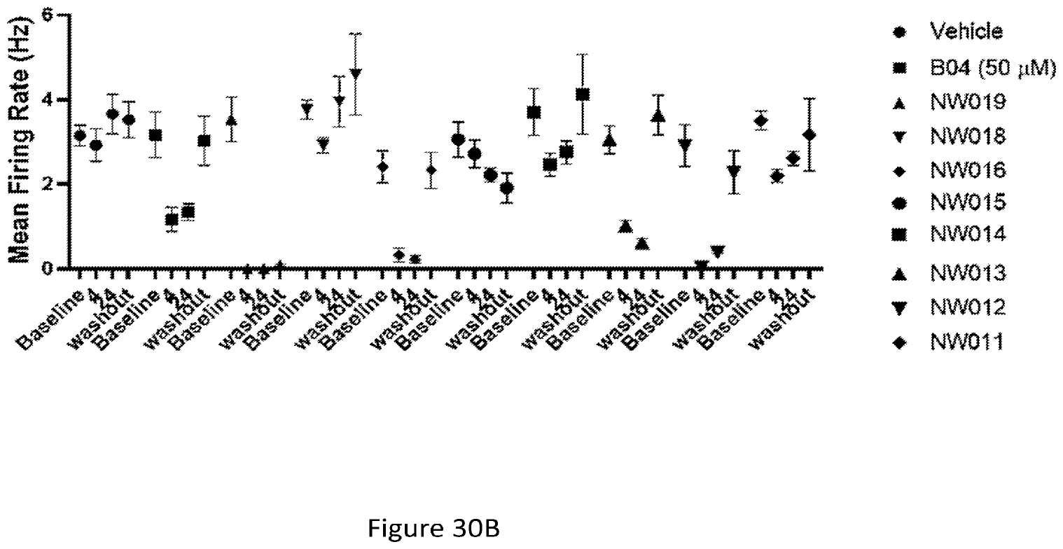

FIGS. 30A and 30B show the neuronal efficacies of selected compounds. compound NW12 is demonstrated to be a reversible inhibitor of neuronal activity, because NW12 inhibited activity robustly and recovered after drug washout.

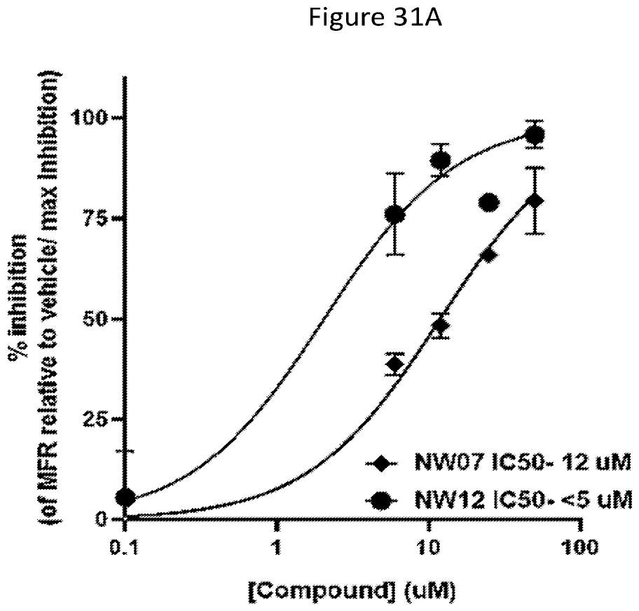

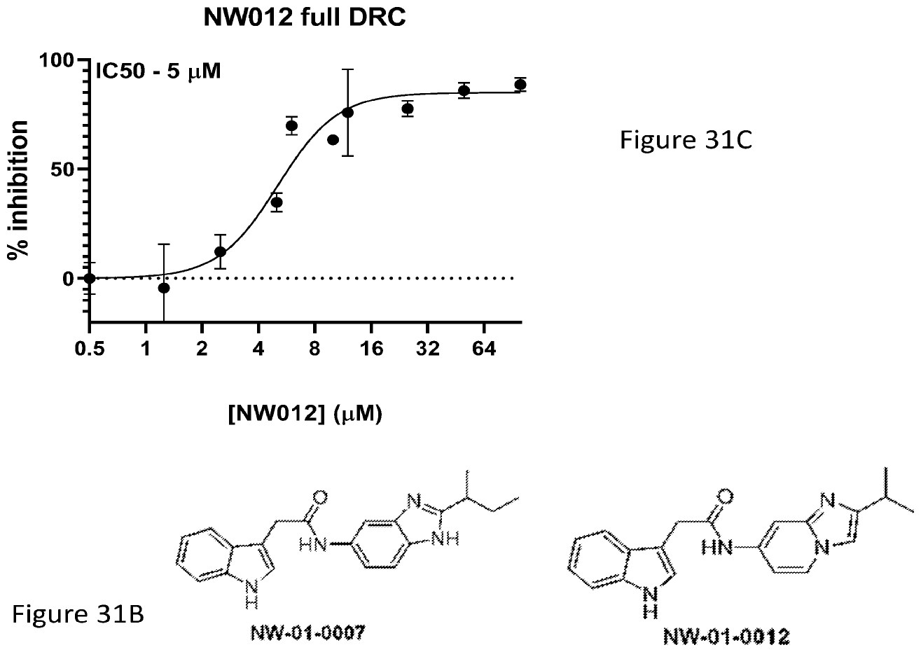

FIGS. 31A-31C illustrate the neuronal efficacies of compounds NW07 and NW12, which are shown to be PP01 analogues of high efficacies. Compound NW07 has an IC₅₀ of 12 μM and compound NW12 has an IC₅₀ lower than 5 μM, indicating that both compounds have enhanced activities compared to compound PP01.

### Example 3—General Synthesis for the Compounds Disclosed Herein

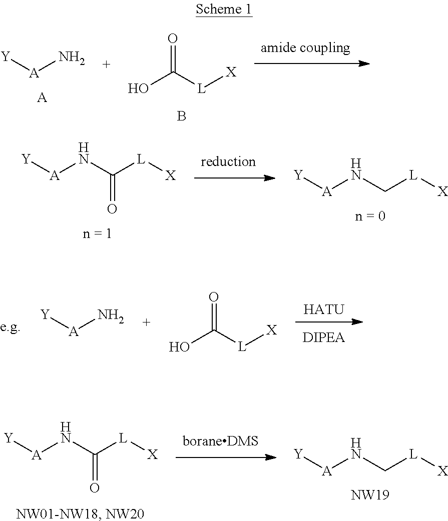

The disclosed compounds that contain amide bonds (n=1) can be synthesized from primary amines A and carboxylic acids B in the presence of known coupling reagents commonly used for the formation of amide bonds. One such procedure employs HATU (1-[bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide) and DIPEA (Hunig's base; N,N-diisopropylethylamine) to acylate the primary amines (e.g. 2-(1H-indol-3-yl)acetic acid) and yield the corresponding amide products. See Scheme 1.

The disclosed compounds that contain a secondary amine moiety (n=0) can be obtained by reducing their corresponding secondary amide precursors in the presence of known reducing agents. For example, borane dimethyl sulfide complex is one of the suitable reducing agents for converting the secondary amide precursors (e.g. 2-(1H-indol-3-yl)-N-(2-isopropyl-1H-benzo[d]imidazol-5-yl)acetamide) to the secondary amine products. See Scheme 1.

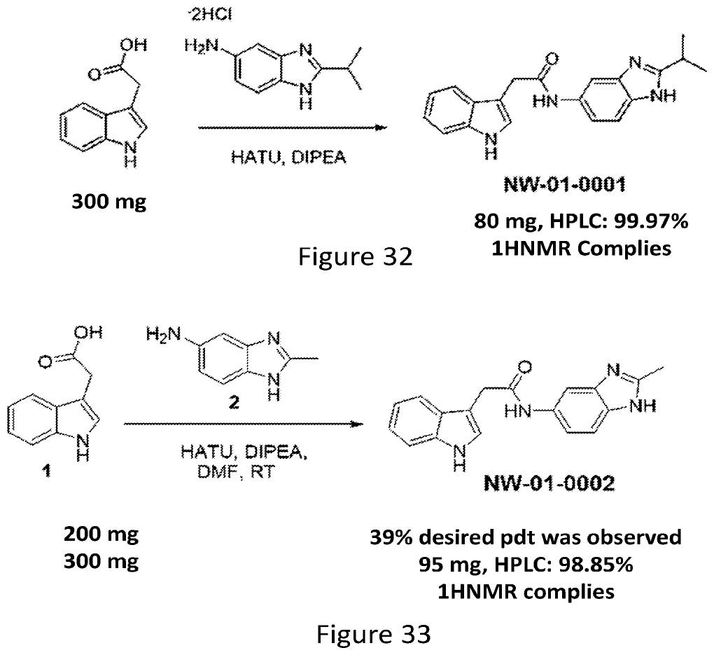

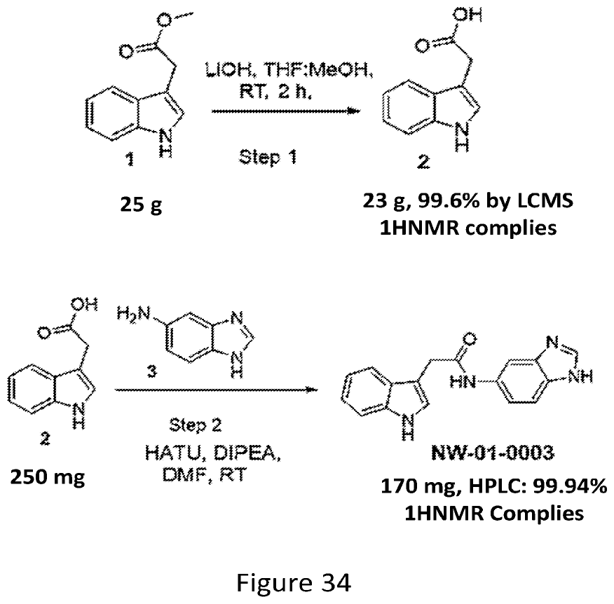

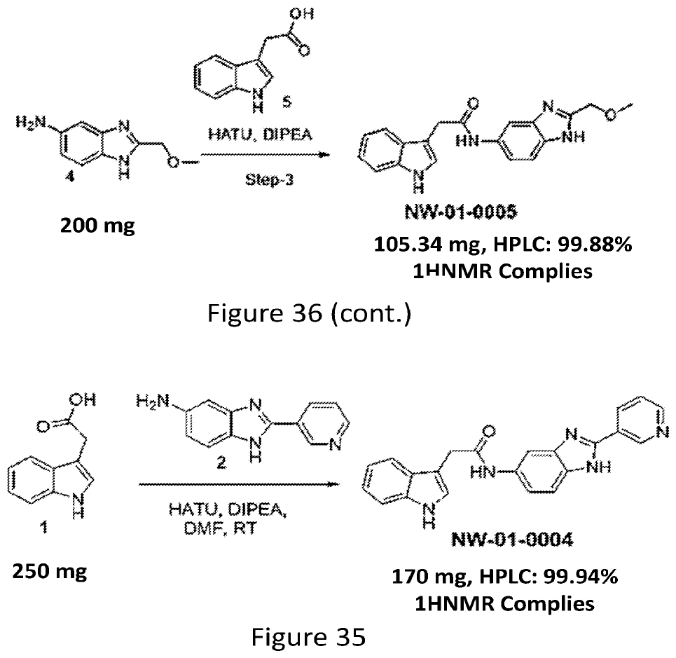

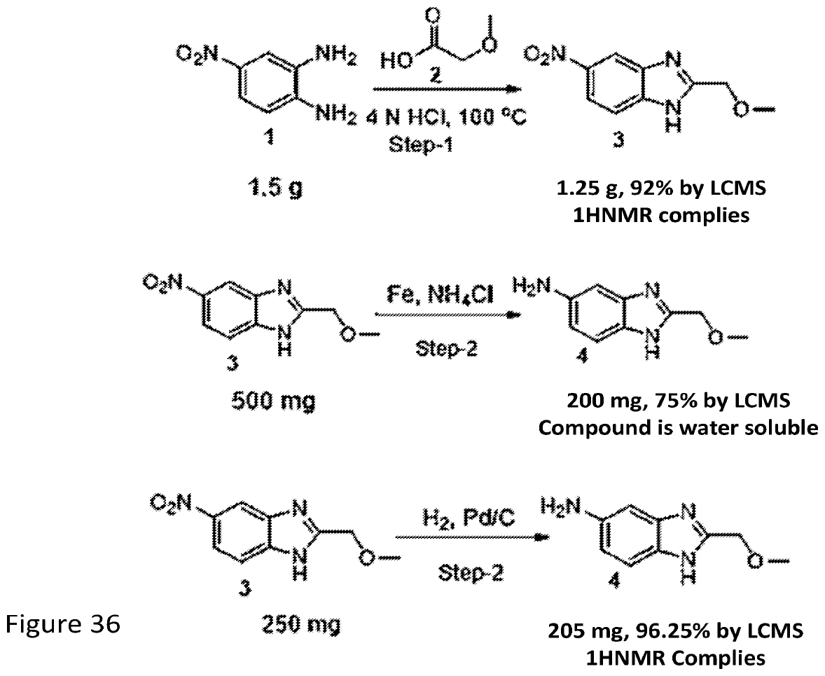

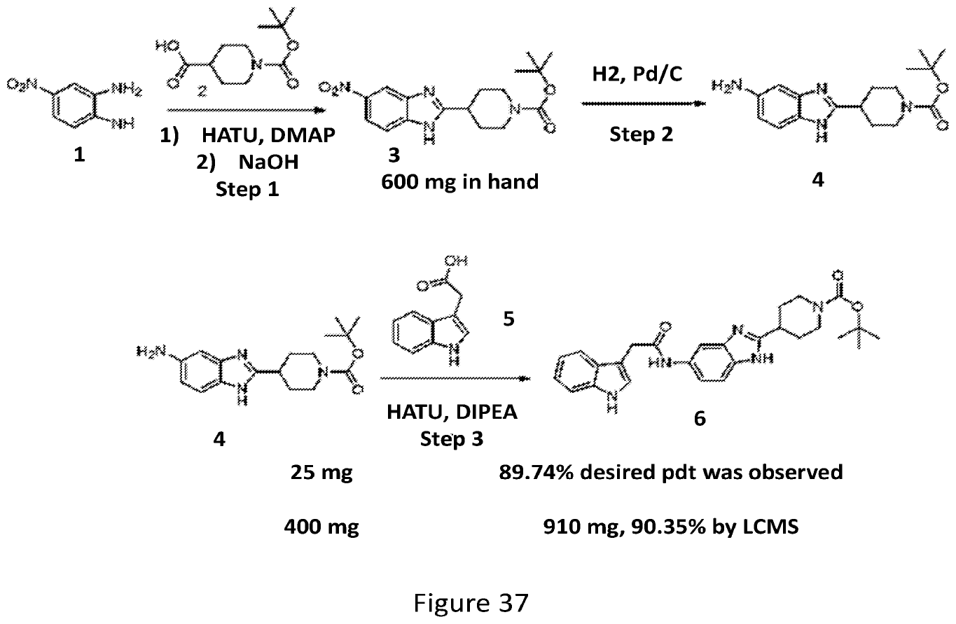

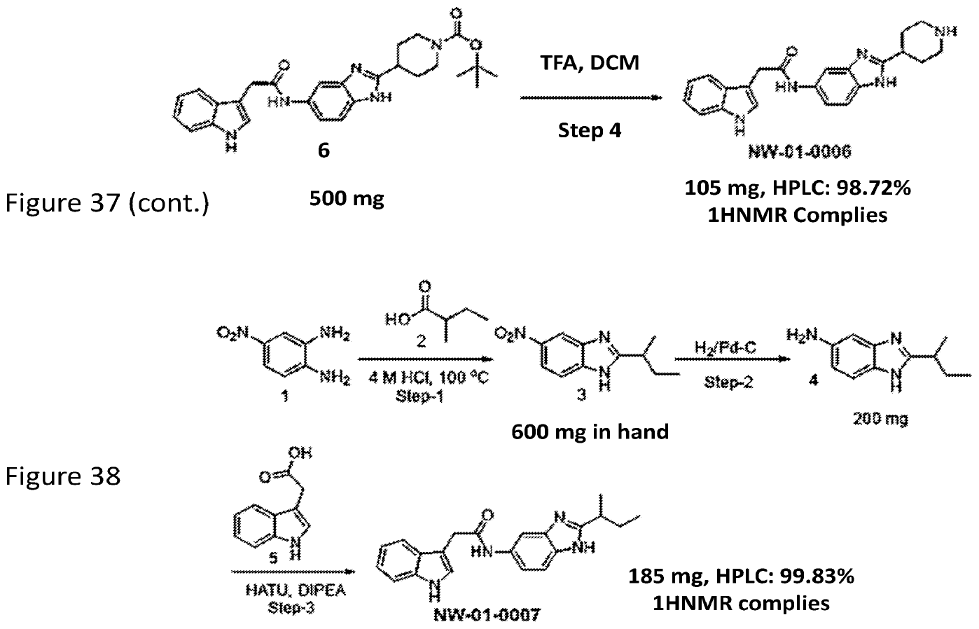

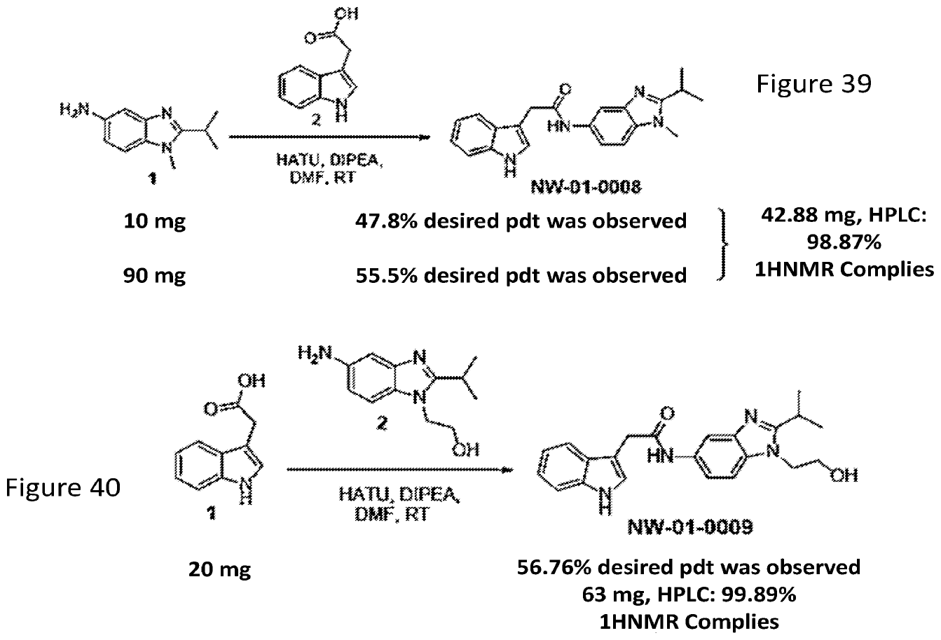

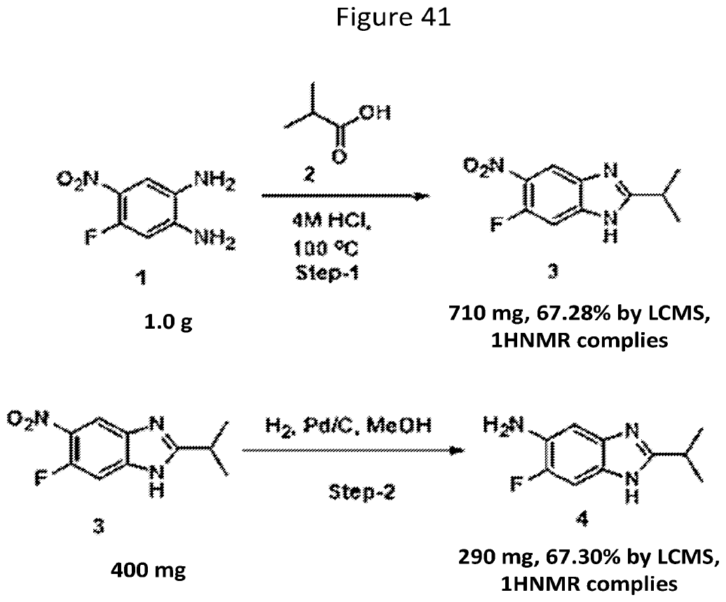

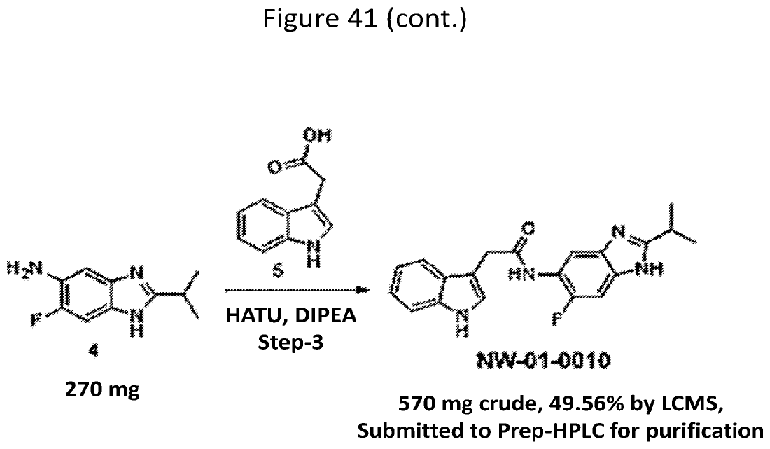

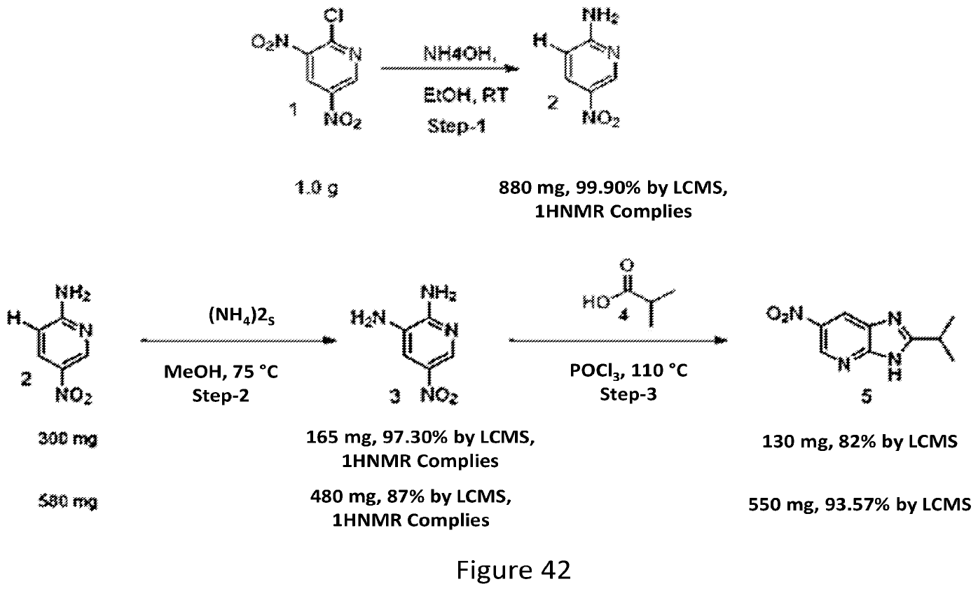

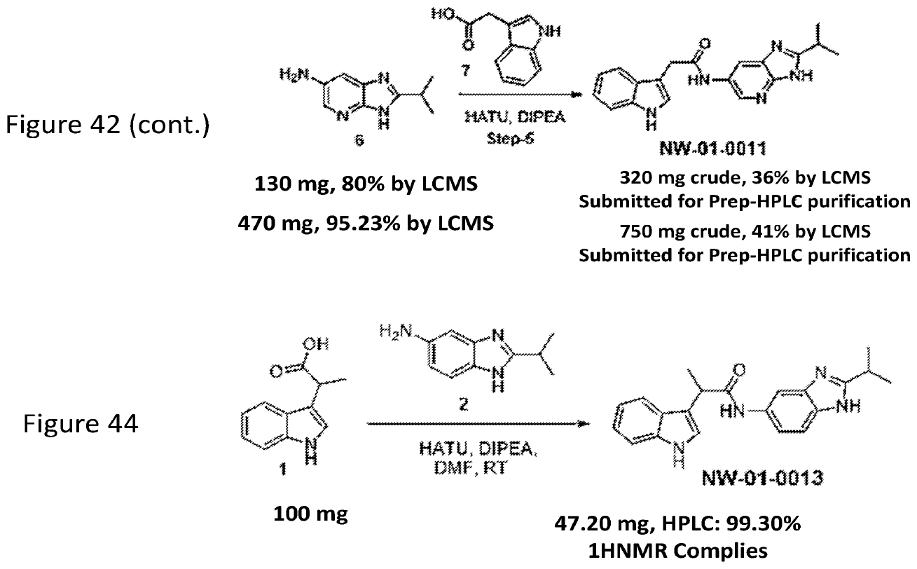

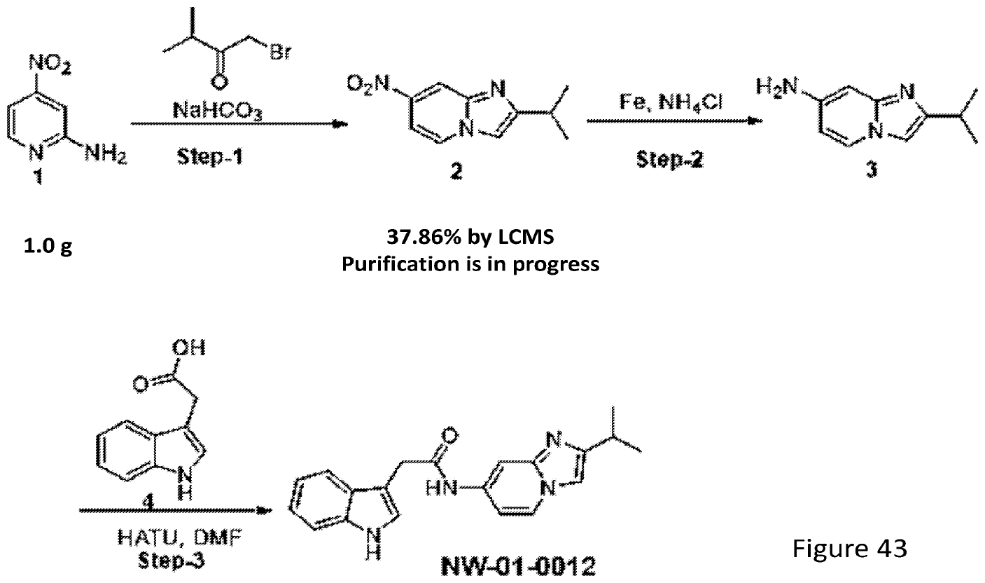

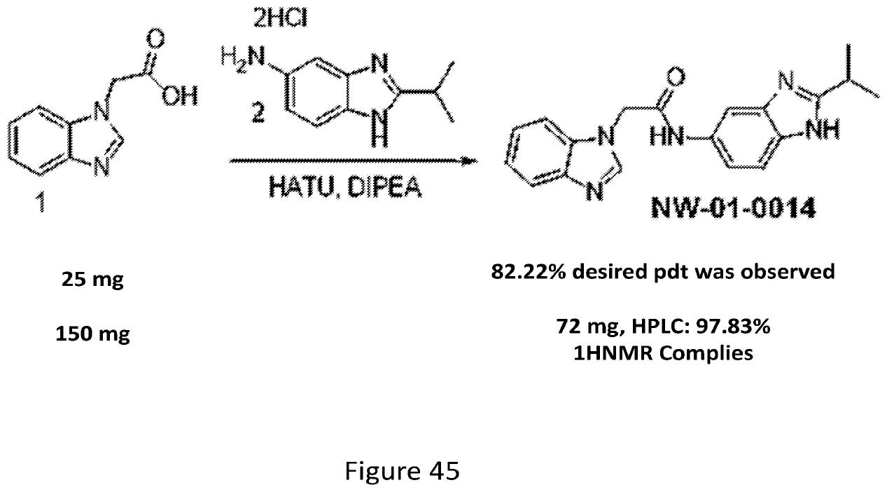

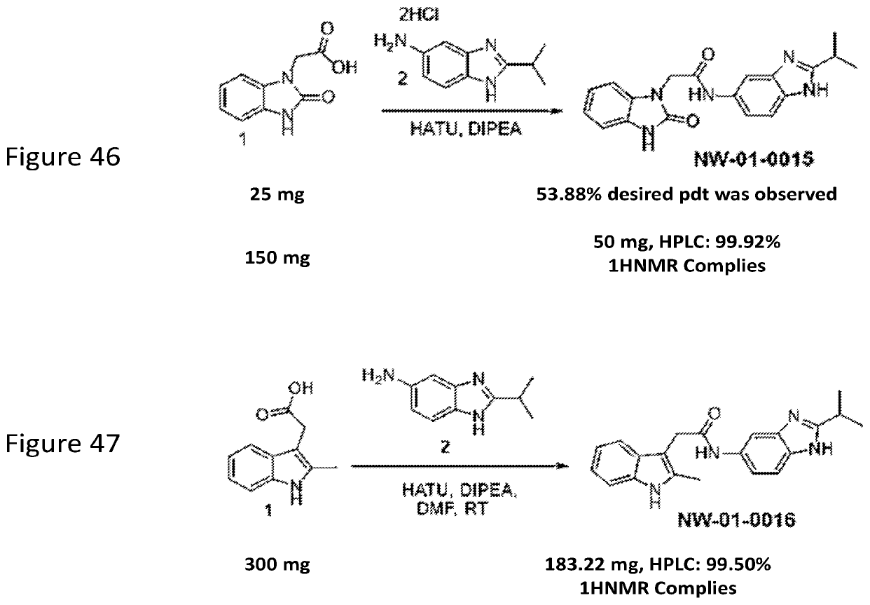

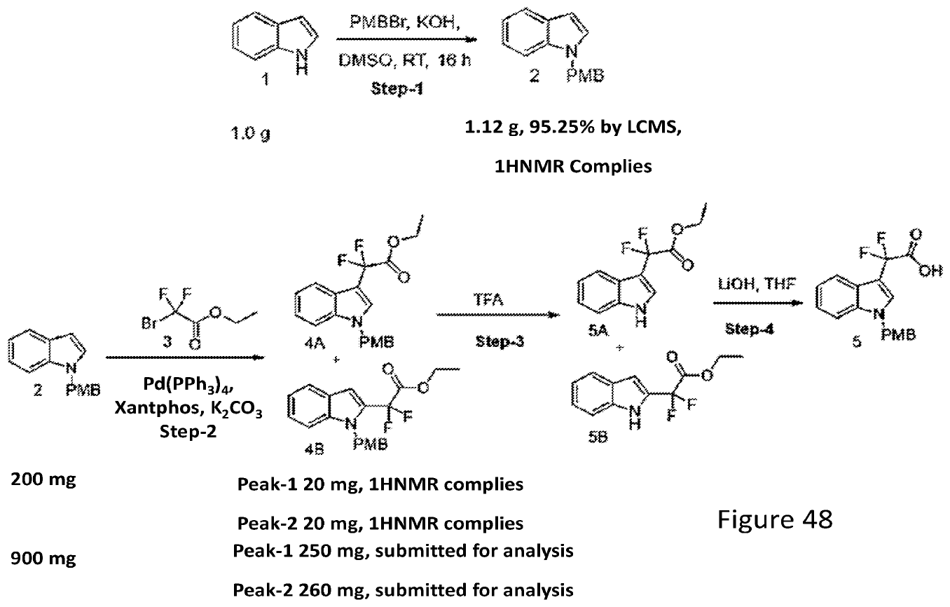

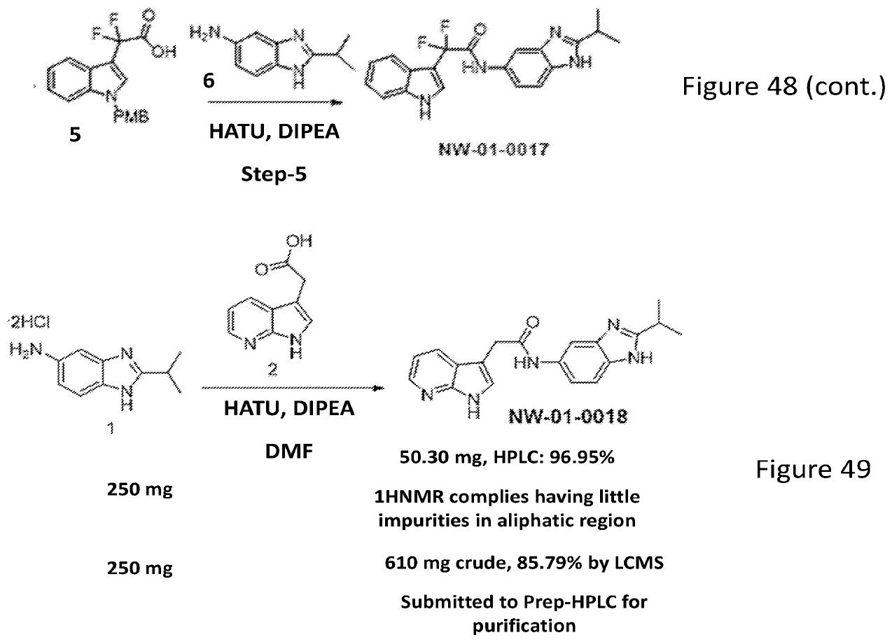

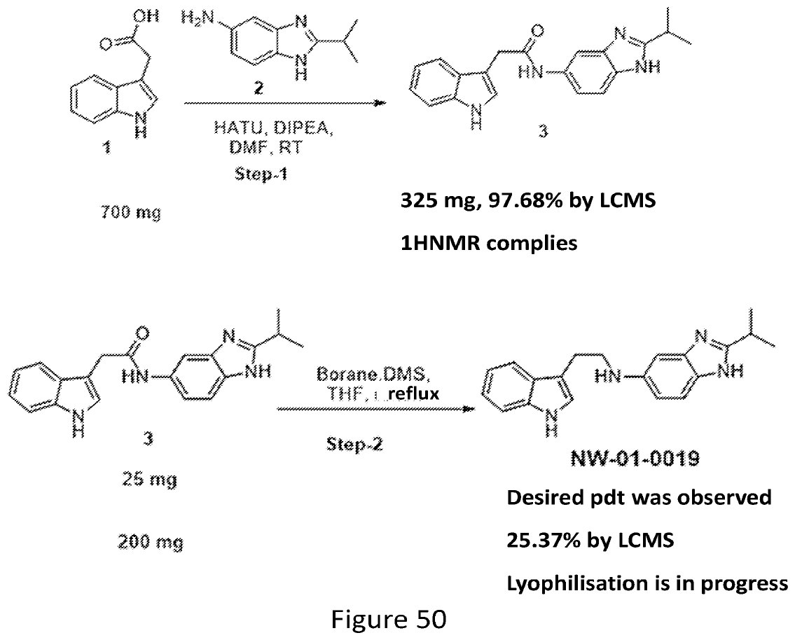

For syntheses of compounds NW-01-0001 to NW-01-0019, see FIGS. 32-50.

In accordance with this disclosure, various other compounds, varied structurally, stereochemically and/or configurationally, are available through such incorporated synthetic procedures and techniques or straight-forward modifications thereof, such modifications as would also be known and understood by those skilled in the art and made aware of this invention, such procedures, techniques and modifications limited only by the commercial or synthetic availability of any corresponding reagent or starting material.

1. A compound of the following formula or a salt or hydrate thereof: 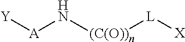
wherein:
Y is selected from hydrogen, alkyl, thiyl, haloalkyl, carboxamido, amido, amino, heteroaryl, alkylene-alkoxy, hydroxyalkyl, and heterocyclyl;
A is an optionally substituted arylene or an optionally substituted heteroarylene selected from 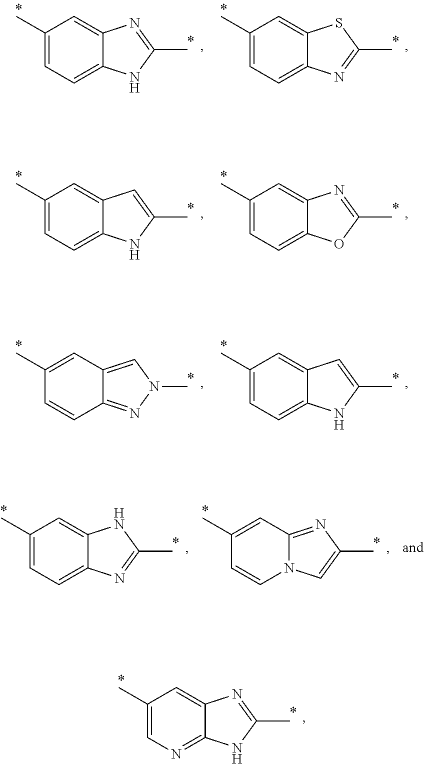
said substituent selected from hydroxyalkyl, halo, and alkyl;
n is 0 or 1;
L is a divalent linker selected from alkylene and halo substituted alkylene; and
X is an optionally substituted heteroaryl selected from 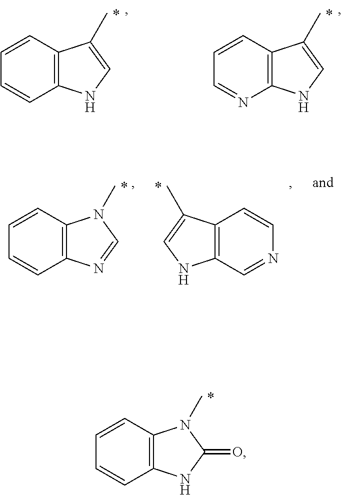
said substituent selected from halo and alkyl.

2. The compound of claim 1, wherein Y is sec-butyl or isopropyl, A is 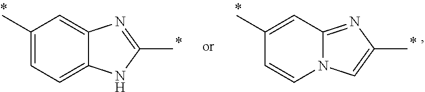
n is 1, L is methylene, and X is

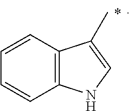

3. The compound of claim 1, wherein L is selected from methylene (—CH₂—), ethylene (—CH₂—CH₂—), 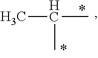
and —CF₂—.

4. The compound of claim 1 of a formula selected from

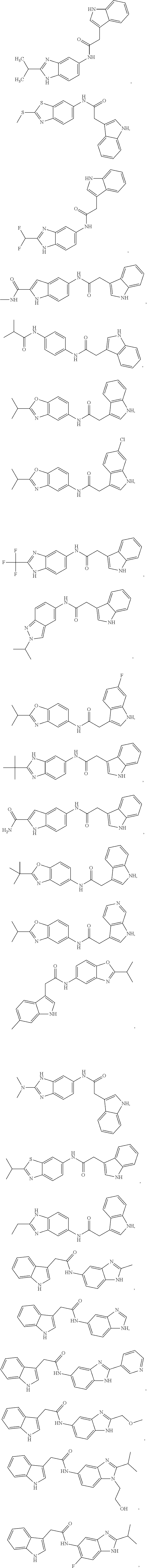

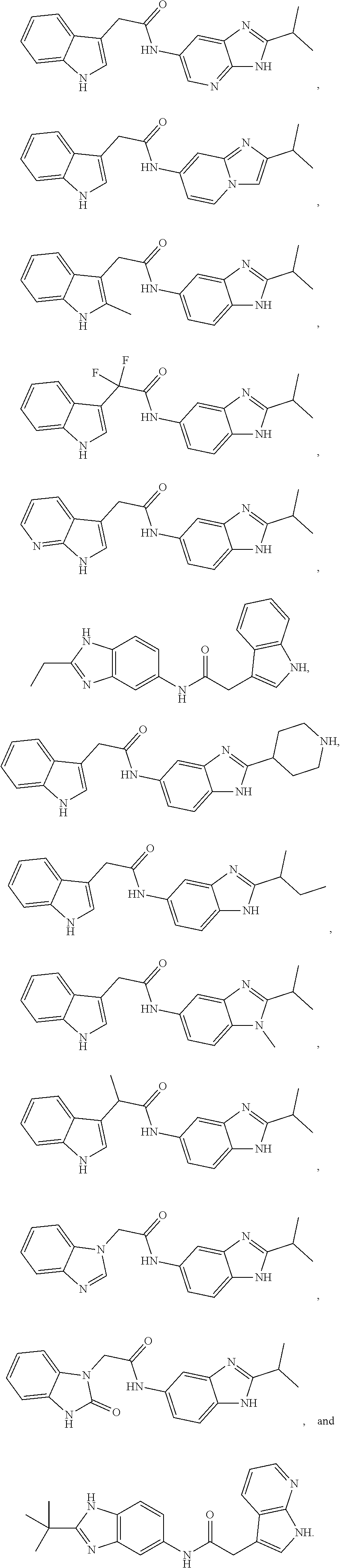

5. The compound of claim 1 of a formula selected from

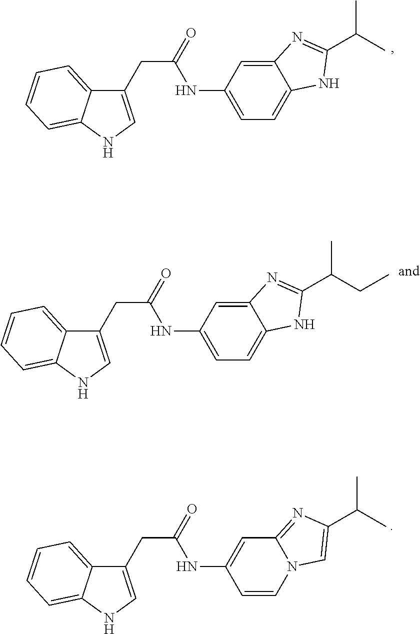 [SMILES: CC(C)c1nc2cc(NC(=O)Cc3c[nH]c4ccccc34)ccc2[nH]1 ; CCC(C)c1nc2cc(NC(=O)Cc3c[nH]c4ccccc34)ccc2[nH]1 ; CC(C)c1cn2ccc(NC(=O)Cc3c[nH]c4ccccc34)cc2n1]

6. The compound of claim 1, wherein the compound is

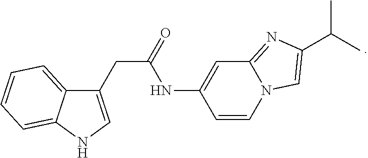 [SMILES: CC(C)c1cn2ccc(NC(=O)Cc3c[nH]c4ccccc34)cc2n1]

7. A pharmaceutical composition comprising the compound of claim 1 and a pharmaceutically suitable carrier, diluent, or excipient.

8. A method for treating and/or preventing a disease or disorder associated with kalirin activity in a subject in need thereof, the method comprising administering to the subject the pharmaceutical composition of claim 7.

9. The method of claim 8, wherein the disease or disorder is neuropathic pain.

10. The method of claim 9, wherein the neuropathic pain is selected from the group consisting of post herpetic neuralgia, reflex sympathetic dystrophy, cancer pain, phantom limb pain, entrapment neuropathy, peripheral neuropathy, trigeminal neuralgia, and any combinations thereof.

11. The method of claim 8, wherein the disease or disorder is chronic pain.

12. The method of claim 11, wherein the chronic pain is selected from the group consisting of headache, nerve damage pain, low back pain, arthritis pain, fibromyalgia pain, and any combinations thereof.

13. The method of claim 8, wherein the disease or disorder is epilepsy.

14. The pharmaceutical composition of claim 7, wherein the compound is selected from

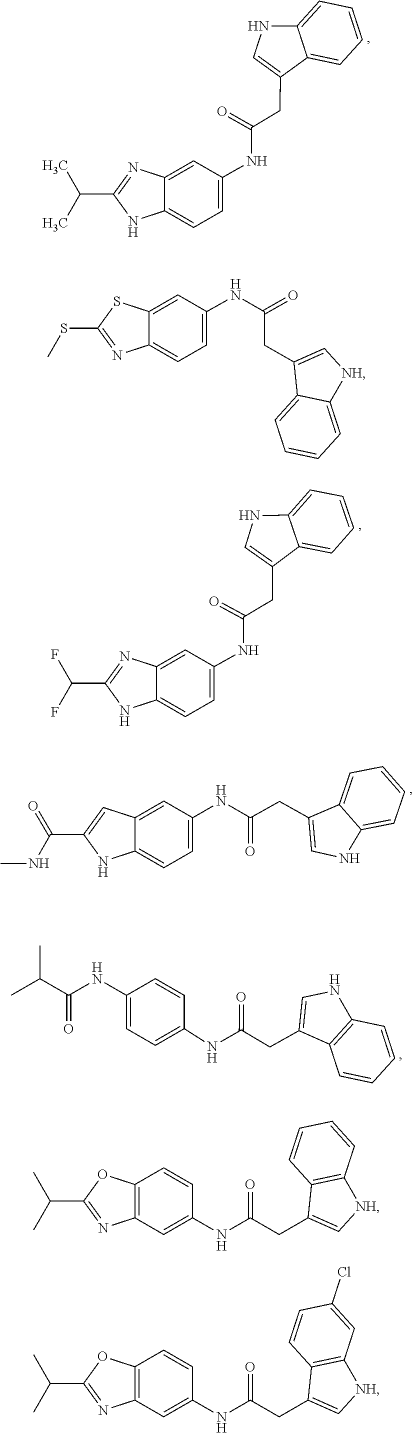

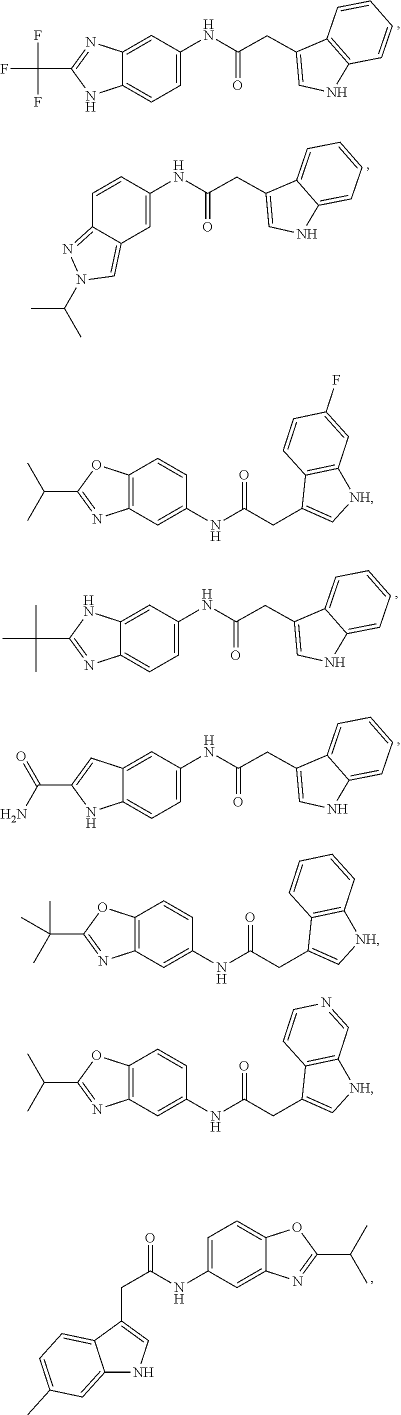

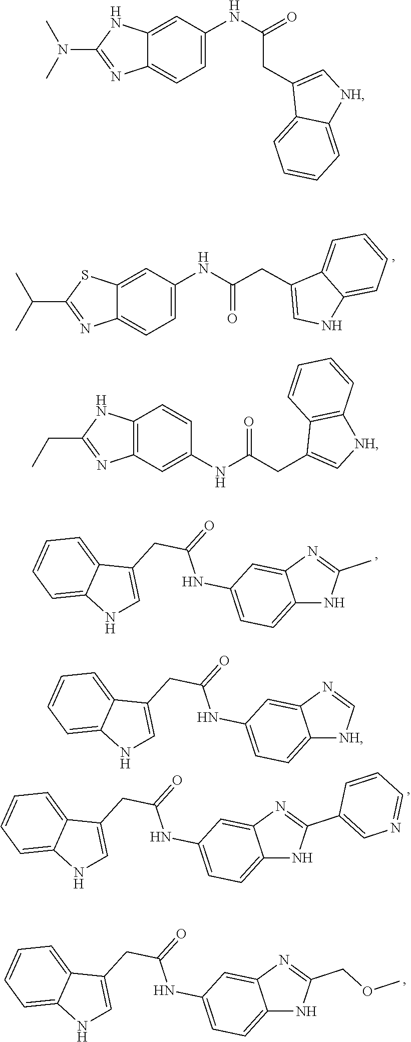

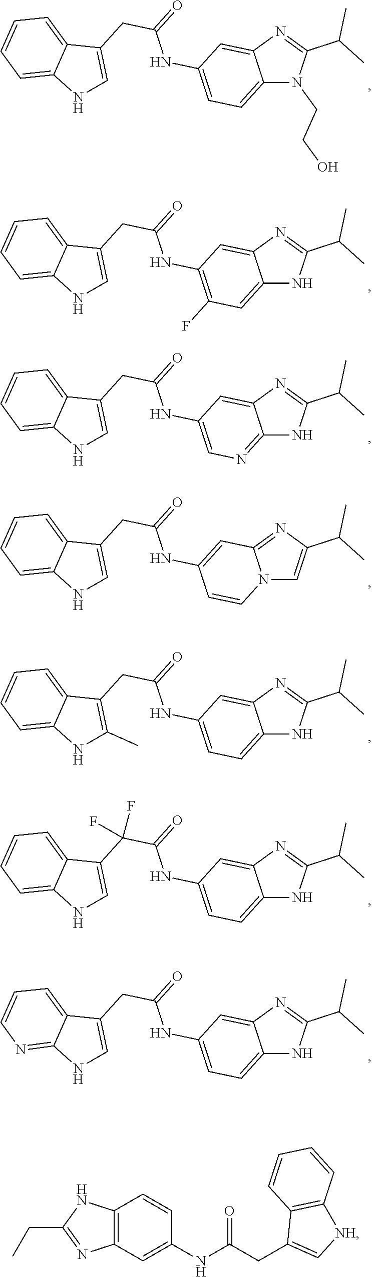

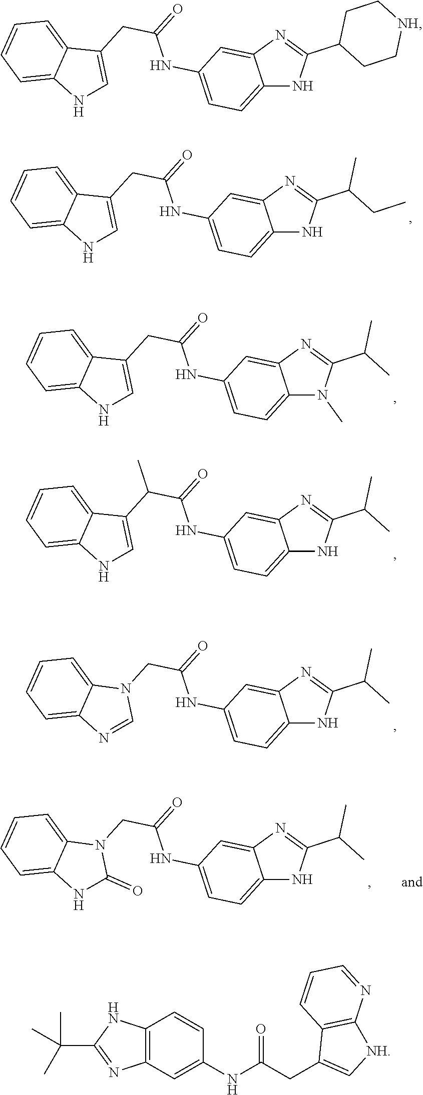

15. The pharmaceutical composition of claim 7, wherein the compound is selected from

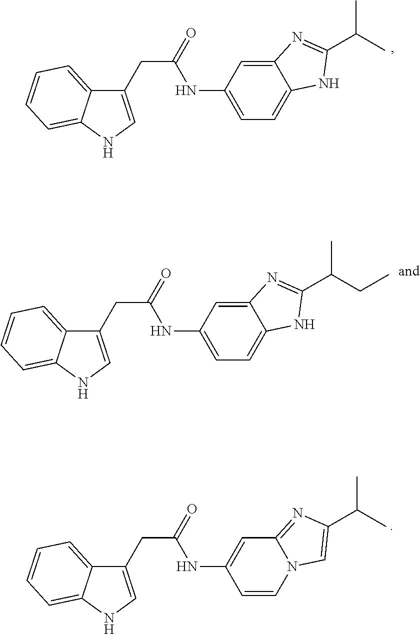 [SMILES: CC(C)c1nc2cc(NC(=O)Cc3c[nH]c4ccccc34)ccc2[nH]1 ; CCC(C)c1nc2cc(NC(=O)Cc3c[nH]c4ccccc34)ccc2[nH]1 ; CC(C)c1cn2ccc(NC(=O)Cc3c[nH]c4ccccc34)cc2n1]

16. The pharmaceutical composition of claim 7, wherein the compound is

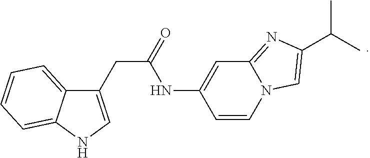 [SMILES: CC(C)c1cn2ccc(NC(=O)Cc3c[nH]c4ccccc34)cc2n1]

17. The method of claim 8, wherein the compound is selected from

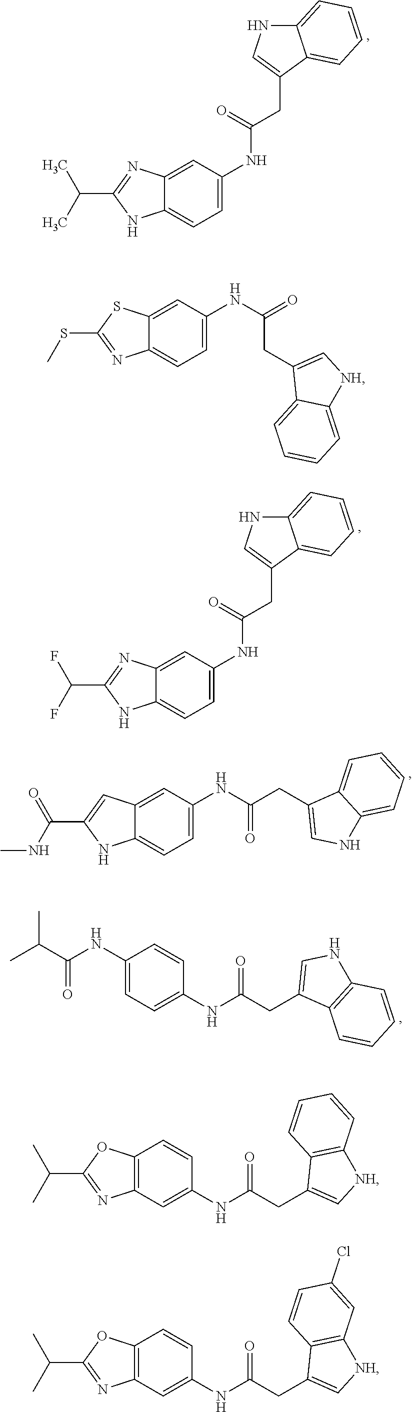

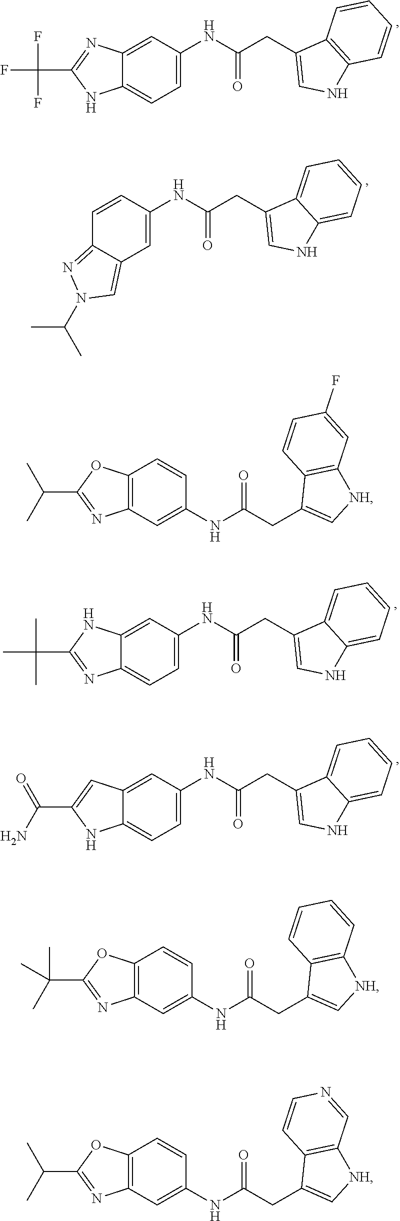

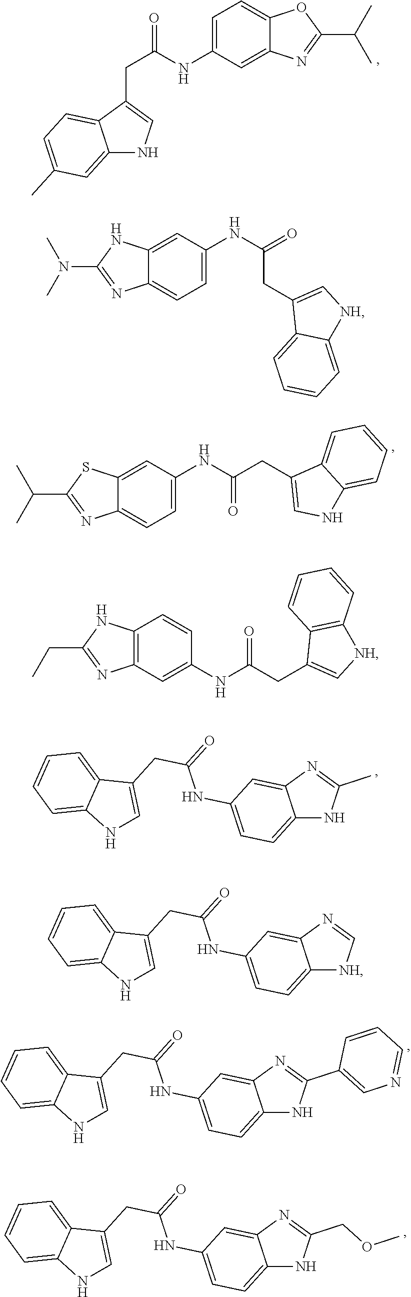

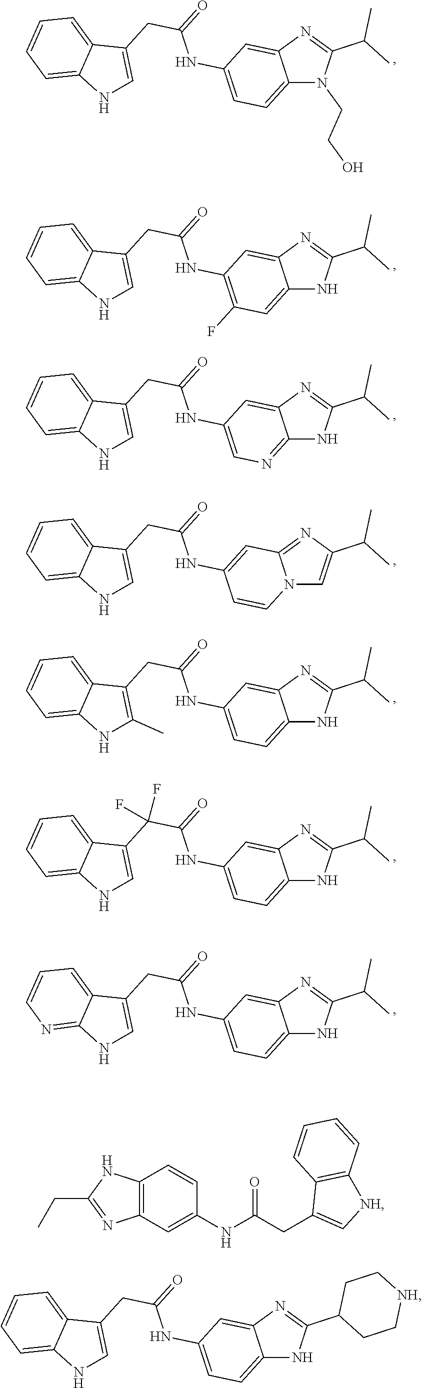

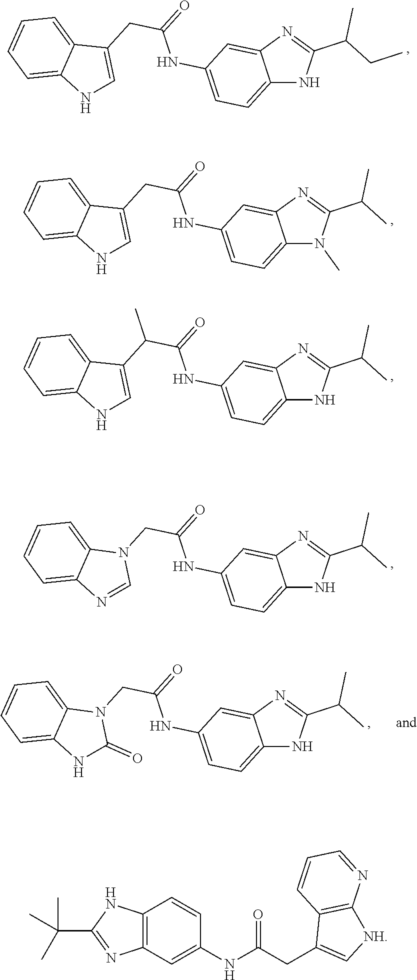

18. The method of claim 8, wherein the compound is selected from

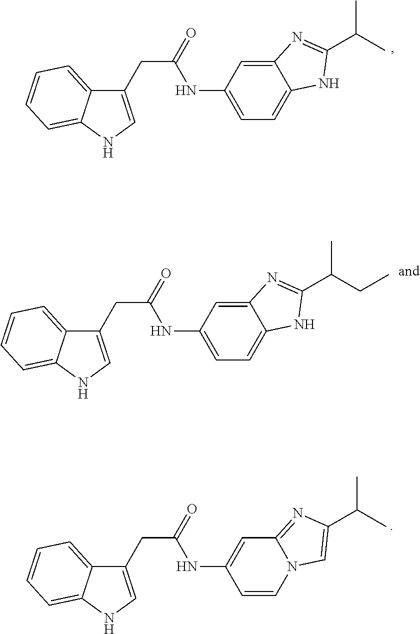 [SMILES: CC(C)c1nc2cc(NC(=O)Cc3c[nH]c4ccccc34)ccc2[nH]1 ; CCC(C)c1nc2cc(NC(=O)Cc3c[nH]c4ccccc34)ccc2[nH]1 ; CC(C)c1cn2ccc(NC(=O)Cc3c[nH]c4ccccc34)cc2n1]

19. The method of claim 8, wherein the compound is 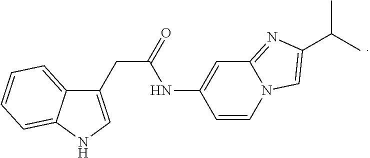 [SMILES: CC(C)c1cn2ccc(NC(=O)Cc3c[nH]c4ccccc34)cc2n1]


SEQUENCE LISTING (8 sequences)

SEQ ID NO: 1 - protein, 290 aa, Homo sapiens
IMAELLQTEKAYVRDLHECLETYLWEMTSGVEEIPPGILNKEHIIFGNIQEIYDFHNNIFLKELEKYEQLPEDVGHCFVTWADKFQMYVTYCKNKPDSNQLILEHAGTFFDEIQQRHGLANSISSYLIKPVQRITKYQLLLKELLTCCEEGKGELKDGLEVMLSVPKKANDAMHVSMLEGFDENLDVQGELILQDAFQVWDPKSLIRKGRERHLFLFEISLVFSKEIKDSSGHTKYVYKNKLLTSELGVTEHVEGDPCKALWSGRTPSSDNKTVLKASNIETKQEWIKNI

SEQ ID NO: 2 - protein, 306 aa, Homo sapiens
VMSELLDTERAYVEELLCVLEGYAAEMDNPLMAHLLSTGLHNKKDVLFGNMEEIYHFHNRIFLRELENYTDCPELVGRCFLERMEDFQIYEKYCQNKPRSESLWRQCSDCPFFQECQRKLDHKLSLDSYLLKPVQRITKYQLLLKEMLKYSRNCEGAEDLQEALSSILGILKAVNDSMHLIAITGYDGNLGDLGKLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEI

SEQ ID NO: 3 - protein, 306 aa, Homo sapiens
VLNELIQTERVYVRELYTVLLGYRAEMDNPEMFDLMPPLLRNKKDILFGNMAEIYEFHNDIFLSSLENCAHAPERVGPCFLERKDDFQMYAKYCQNKPRSETIWRKYSECAFFQECQRKLKHRLRLDSYLLKPVQRITKYQLLLKELLKYSKDCEGSALLKKALDAMLDLLKSVNDSMHQIAINGYIGNLNELGKMIMQGGFSVWIGHKKGATKMKDLARFKPMQRHLFLYEKAIVFCKRRVESGEGSDRYPSYSFKHCWKMDEVGITEYVKGDNRKFEIWYGEKEEVYIVQASNVDVKMTWLKEI

SEQ ID NO: 4 - protein, 290 aa, Homo sapiens
IMAELIQTEKAYVRDLRECMDTYLWEMYSGVEEIPPGIVNKELIIFGNMQEIYEFHNNIFLKELEKYEQLPEDVGHCFVTWADKFQMYVTYCKNKPDSTQLILEHAGSYFDEIQQRHGLANSISSYLIKPVQRITKYQLLLKELLTCCEEGKGEIKDGLEVMLSVPKRANDAMHLSMLEGFDENIESQGELILQESFQVWDPKTLIRKGRERHLFLFEMSLVFSKEVKDSSGRSKYLYKSLFTSELGVTEHVEGDPCKFALWVGRTPTSDNKIVLKASSIENKQDWIKHI

SEQ ID NO: 5 - protein, 191 aa, Homo sapiens
MQTIKCVVVGDGACGKTCLLISTYYNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 6 - protein, 192 aa, Homo sapiens
MQAIKCVVVGDGACGKTCLLISYTTNAFPGEYIPTVFDNYSANVMVDGKPVNLGLWDTAGQEDYDRLRPLSYPQTDVFLICFSLVSPASFENVRAKWYPEVRJJCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 7 - protein, 193 aa, Homo sapiens
MAAIRKKLVIVGDGACGKTCKKIVFSKDQFPEVYVPTVFENTVADIEVDGKQVELALWDTAGQEDTDRLRPLSYPDTDVILMCFSIDSPDSLENIPEKQTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 8 - protein, 189 aa, Homo sapiens
MTEYKLVVVGAGGVGKSALTIQLIQNHFVDEYDPTIEDSYRKQVVIDGETQLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS